SEQUENCE LISTING (32 sequences)

SEQ ID NO: 1 - DNA, 21 bp, Artificial Sequence
  ATIII RT forward primer
gcttttgctatgaccaagctg

SEQ ID NO: 2 - DNA, 21 bp, Artificial Sequence
  ATIII RT Reverse primer
tgcttcactgccttcttcatt

SEQ ID NO: 3 - DNA, 21 bp, Artificial Sequence
  IL1F7 RT Forward primer
aaacccgaagaaattcagcat

SEQ ID NO: 4 - DNA, 21 bp, Artificial Sequence
  IL1F7 RT reverse primer
cccacctgagccctataaaag

SEQ ID NO: 5 - DNA, 21 bp, Artificial Sequence
  IL25 RT Forward primer
ttcctacaggtggttgcattc

SEQ ID NO: 6 - DNA, 21 bp, Artificial Sequence
  IL25 RT Reverse primer
cgcctgtagaagacagtctgg

SEQ ID NO: 7 - DNA, 21 bp, Artificial Sequence
  IL25 RT Reverse primer
aatcaaggctcagcaatctca

SEQ ID NO: 8 - DNA, 21 bp, Artificial Sequence
  VBP RT Reverse primer
catctttgtttgtgggcaact

SEQ ID NO: 9 - DNA, 21 bp, Artificial Sequence
  IL25 Receptor RT Forward primer
agaggccttccagactcagac

SEQ ID NO: 10 - DNA, 21 bp, Artificial Sequence
  IL25 Receptor RT Reverse primer
aaacccgatgatagtgctgtg

SEQ ID NO: 11 - DNA, 23 bp, Artificial Sequence
  Alpha-tubulin Forward primer
tgacctgacagaattccagacca

SEQ ID NO: 12 - DNA, 23 bp, Artificial Sequence
  Alpha-tubulin reverse primer
gcattgacatctttgggaaccac

SEQ ID NO: 13 - DNA, 21 bp, Artificial Sequence
  IL25 Receptor siRNA Forward sequence
cgcgagcuucaguggugautt

SEQ ID NO: 14 - DNA, 21 bp, Artificial Sequence
  IL25 Receptor siRNA Reverse Sequence
aucaccacugaagcucgcgtt

SEQ ID NO: 15 - protein, 177 aa, Homo sapiens
MRERPRLGEDSSLISLFLQVVAFLAMVMGTHTYSHWPSCCPSKGQDTSEELLRWSTVPVPPLEPARPNRHPESCRASEDGPLNSRAISPWRYELDRDLNRLPQDLYHARCLCPHCVSLQTGSHMDPRGNSELLYHNQTVFYRRPCHGEKGTHKGYCLERRLYRVSLACVCVRPRVMG

SEQ ID NO: 16 - DNA, 1334 bp, Homo sapiens
ggcttgctgaaaataaaatcaggactcctaacctgctccagtcagcctgcttccacgaggcctgtcagtcagtgcccgacttgtgactgagtgtgcagtgcccagcatgtaccaggtcagtgcagagggctgcctgagggctgtgctgagagggagaggagcagagatgctgctgagggtggagggaggccaagctgccaggtttggggctgggggccaagtggagtgagaaactgggatcccagggggagggtgcagatgagggagcgacccagattaggtgaggacagttctctcattagccttttcctacaggtggttgcattcttggcaatggtcatgggaacccacacctacagccactggcccagctgctgccccagcaaagggcaggacacctctgaggagctgctgaggtggagcactgtgcctgtgcctcccctagagcctgctaggcccaaccgccacccagagtcctgtagggccagtgaagatggacccctcaacagcagggccatctccccctggagatatgagttggacagagacttgaaccggctcccccaggacctgtaccacgcccgttgcctgtgcccgcactgcgtcagcctacagacaggctcccacatggacccccggggcaactcggagctgctctaccacaaccagactgtcttctacaggcggccatgccatggcgagaagggcacccacaagggctactgcctggagcgcaggctgtaccgtgtttccttagcttgtgtgtgtgtgcggccccgtgtgatgggctagccggacctgctggaggctggtccctttttgggaaacctggagccaggtgtacaaccacttgccatgaagggccaggatgcccagatgcttggcccctgtgaagtgctgtctggagcagcaggatcccgggacaggatggggggctttggggaaaacctgcacttctgcacattttgaaaagagcagctgctgcttagggccgccggaagctggtgtcctgtcattttctctcaggaaaggttttcaaagttctgcccatttctggaggccaccactcctgtctcttcctcttttcccatcccctgctaccctggcccagcacaggcactttctagatatttcccccttgctggagaagaaagagcccctggttttatttgtttgtttactcatcactcagtgagcatctactttgggtgcattctagtgtagttactagtcttttgacatggatgattctgaggggaagctgttattgaatgtatagagatttatccaaataaatatctttatttaaaaatgaaaaaaaaaaaaaaaaaaaa

SEQ ID NO: 17 - DNA, 21 bp, Artificial Sequence
  Luc siRNA Sense strand
cguacgcggaauacuucgatt

SEQ ID NO: 18 - DNA, 21 bp, Artificial Sequence
  Luc Antisense siRNA strand
ucgaaguauuccgcguacgtt

SEQ ID NO: 19 - RNA, 21 nt, Artificial sequence
  FADD  siRNA Sense strand (target nt 663-683; gi 22219473)
acaccaagaucgacagcauuu

SEQ ID NO: 20 - RNA, 21 nt, Artificial Sequence
  FADD siRNA Antisense strand (target nt 663-683; gi 22219473)
augcugucgaucuugguguuu

SEQ ID NO: 21 - RNA, 21 nt, Artificial Sequence
  TRADD siRNA Sense strand (target nt 542-562; gi 115387096)
uggaggaugcgcugcgaaauu

SEQ ID NO: 22 - RNA, 21 nt, Artificial sequence
  TRADD siRNA Antisense strand (target nt 542-562; gi 115387096)
uuucgcagcgcauccuccauu

SEQ ID NO: 23 - DNA, 21 bp, Artificial Sequence
  IL17RB #1 siRNA Sense strand, target nt 729-749 from TSS (gi112382255)
gaaacaaacgcgagcuucatt

SEQ ID NO: 24 - DNA, 21 bp, Artificial Sequence
  IL17RB #1 siRNA Antisense Strand, target nt 729-749 from TSS (gi112382255)
ugaagcucgcguuuguuuctt

SEQ ID NO: 25 - DNA, 21 bp, Artificial Sequence
  IL17RB #2 siRNA Sense  Strand; targets nt 721-741 from TSS (gi112382255)
aucaccacugaagcucgcgtt

SEQ ID NO: 26 - DNA, 19 bp, Artificial Sequence
  IL17RB #2 siRNA Antisense Strand, targets nt 721-741 from TSS (gi112382255)
gaaacaaacgcgagcuuca

SEQ ID NO: 27 - DNA, 21 bp, Artificial Sequence
  RT-PCR primer for Alpha-tubulin product ˜250 bp; Sense strand
ugaagcucgcguuuguuuctt

SEQ ID NO: 28 - DNA, 23 bp, Artificial Sequence
  RT-PCR primer for Alpha-tubulin product ˜250 bp; Antisense strand
tgacctgacagaattccagacca

SEQ ID NO: 29 - DNA, 23 bp, Artificial Sequence
  IL17RB siRNA product ˜350 bp; Sense strand, target is 354-374 nt from TSS (gi112382255)
gcattgacatctttgggaaccac

SEQ ID NO: 30 - DNA, 21 bp, Artificial Sequence
  IL17RB siRNA product ˜350 bp; Antisense [676-698 nt from TSS (gi112382255)]
aaacccgatgatagtgctgtg

SEQ ID NO: 31 - DNA, 19 bp, Artificial Sequence
  siRNA Sense strand sequence targets the original endogenously produced wild-type IL17RB mRNA  (nt 731-749) from TSS (gi112382255)
cgcgagcttcagtggtgat

SEQ ID NO: 32 - DNA, 19 bp, Artificial Sequence
  siRNA sense strand targets exogenously overexpressed mutant form of IL17RB mRNA at mutated nt 735, 739-741 from TSS (gi112382255)  mutations in the region targeted by IL17RB siRNA #1
cgcgggctagcgtggtgat

(19) United States
(12) Patent Application Publication
(10) Pub. No.: US 2013/0052157 A1
(43) Pub. Date: Feb. 28, 2013

(54) USE OF IL-17E FOR CANCER TREATMENT

(71) Applicants: Saori Furuta, Emeryville, CA (US); Mina J. Bissell, Berkeley, CA (US); Wen-Hwa Lee, Newport Coast, CA (US); Eva Yhp Lee, Newport Coast, CA (US)

(72) Inventors: Saori Furuta, Emeryville, CA (US); Mina J. Bissell, Berkeley, CA (US); Wen-Hwa Lee, Newport Coast, CA (US); Eva Yhp Lee, Newport Coast, CA (US)

(73) Assignee: The Regents of the University of California, Oakland, CA (US)

(21) Appl. No.: 12/445,457

(22) Filed: Apr. 13, 2009

Related U.S. Application Data
(63) Continuation-in-part of application No. PCT/US2007/081395, filed on Oct. 15, 2007.
(60) Provisional application No. 60/851,446, filed on Oct. 13, 2006.
(60) Provisional application No. 60/972,111, filed on Sep. 13, 2007.

(51) Int. Cl. A61K 38/20; A61P 35/00; C12N 5/09; C07K 14/54; C07H 21/04

(52) U.S. Cl. 424/85.2; 530/351; 536/23.5; 435/375

(57) ABSTRACT

The present invention provides methods, kits, and compositions for treating cancer with cytotoxic agents. Preferably, the cytotoxic agents are selected from: IL-25, BMP1O, FGF1 1, VDBP, ATIII and IL1-F7, and any combination thereof. In other preferred embodiments, the cancer is breast cancer. These agents can be supplied, for example, as proteins or as part of nucleic acid expression vector.

The present application claims priority to both U.S. Provisional Application Ser. No. 60/851,446, filed Oct. 13, 2006, and U.S. Provisional Application Ser. No. 60/972,111, filed Sep. 13, 2007, both of which are herein incorporated by reference.

This invention was made with government support under grant number R01CA94170 awarded by the National Institute of Health. The government has certain rights in the invention.

### FIELD OF THE INVENTION

The present invention provides methods, kits, and compositions for treating cancer with cytotoxic agents. Preferably, the cytotoxic agents are selected from: IL-25 (IL-17E), BMP10, FGF11, VDBP, ATIII and IL1-F7, and any combination thereof. In other preferred embodiments, the cancer is breast cancer.

### BACKGROUND OF THE INVENTION

Breast cancer is the most common female malignancy in most industrialized countries as it is estimated to affect about 10% of the female population during their lifespan. Although its mortality has not increased along with its incidence, due to earlier diagnosis and improved treatment, it is still one of the predominant causes of death in middle aged women.

The primary treatment for breast cancer is surgery, either alone or combined with systemic adjuvant therapy (hormonal or cytotoxic) and/or post operative irradiation. Most patients are cured with these treatments, but approximately 25-30% of women with node negative disease and at least 50-60% of women with positive nodes, who appear to be disease free after locoregional treatment, will relapse and need treatment for their metastatic disease. Thus, breast cancer is a significant and growing problem in oncology.

### SUMMARY OF THE INVENTION

The present invention provides methods, kits, and compositions for treating cancer with cytotoxic agents. Preferably, the cytotoxic agents are selected from: IL-25, BMP10, FGF11, VDBP, ATIII and IL1-F7, and any combination thereof. In other preferred embodiments, the cancer is breast cancer. These agents can be supplied, for example, as proteins or as part of nucleic acid expression vector (e.g., an adeno-virus encoding one of the cytotoxic agents).

In some embodiments, the present invention provides methods comprising contacting cancer cells (e.g., breast cancer cells) with a therapeutic amount of at least one cytotoxic factor selected from the group consisting of: IL-25, BMP10, FGF11, VDBP, ATIII and IL1-F7. In certain embodiments, the cytotoxic agent suppresses proliferation of the breast cancer cells. In other embodiments, the agent does not suppress differentiation of the breast cancer cells. In particular embodiments, the cytotoxic agent is IL-25. In other embodiments, the cytotoxic agent is BMP10. In some embodiments, the cytotoxic agent is FGF11. In further embodiments, the cytotoxic agent is VDBP.

In particular embodiments, the contacting kills at least a portion of the breast cancer cells. In other embodiments, contacting is performed in vivo (e.g., a patient is treated) or in vitro. In further embodiments, the at least one cytotoxic agent includes at least two of the cytotoxic agents. In some embodiments, the at least one cytotoxic agent includes at least 3 or 4, or 5 or 6 of the cytotoxic agents.

In other embodiments, the present invention provides compositions comprising: a) a known breast cancer treatment agent (e.g., HERCEPTIN, Cisplatin, etc.) and b) at least one cytotoxic agent selected from the group consisting of: IL-25, BMP10, FGF11, VDBP, ATIII and IL1-F7.

In further embodiments, the present invention provides kits comprising: a) a known breast cancer treatment agent and b) at least one cytotoxic agent selected from the group consisting of: IL-25, BMP10, FGF11, VDBP, ATIII and IL1-F7.

In certain embodiments, the present invention provides methods comprising: treating breast cancer cells with a size fractioned conditioned medium (CDMD) collected from differentiating normal MECs (mammary epithelial cells), wherein the size fractioned conditioned medium is enriched for the 10-50 kDa fraction. In some embodiments, the size fractioned condition medium is enriched at least 2-fold (or 3-fold, 4-fold . . . 10-fold . . . 100-fold . . . 1000-fold or more) compared to un-enriched conditioned medium. In other embodiments, the breast cancer cells are differention-defective.

In some embodiments, the present invention provides methods comprising contacting cancer cells (e.g., breast cancer cells) in a patient with a therapeutic amount of an agent configured to: i) bind an IL-25 receptor, and ii) activate caspase mediated apoptosis, wherein said cancer cells over-express IL-25 receptor compared to non-cancer breast cells.

In further embodiments, the present invention provides methods comprising contacting cancer cells (e.g., breast cancer cells) in a patient with a nucleic acid vector (e.g., AAV) configured to express an agent configured to: i) bind an IL-25 receptor, and ii) activate caspase mediated apoptosis, wherein said breast cancer cells over-express IL-25 receptor compared to non-cancer breast cells.

In particular embodiments, the agent comprises IL-25 protein. In other embodiments, the agent comprises an IL-25 variant. In some embodiments, the IL-25 variant is selected from the group consisting of: an IL-25 truncated protein; and IL-25 mutant with substituted, deleted, or additional amino acids. In particular embodiments, the agent is an IL-25 mimetic. In some embodiments, the agent is a monoclonal antibody or antibody fragment. In further embodiments, the monoclonal antibody or antibody fragment is a chimeric, humanized, or human antibody or fragment thereof.

In certain embodiments, the agent suppresses proliferation of the cancer cells. In other embodiments, the agent does not suppress differentiation of the cancer cells. In particular embodiments, the contacting kills at least a portion of the cancer cells.

In certain embodiments, the present invention provides compositions comprising: a) a known breast cancer treatment agent and b) at least one agent configured to: i) bind an IL-25 receptor, and ii) activate caspase mediated apoptosis, in cancer cells (e.g., breast cancer cells) that over-express IL-25 receptor compared to non-cancer cells. In other embodiments, the present invention provides kits comprising: a) a known breast cancer treatment agent and b) at least one agent configured to: i) bind an IL-25 receptor, and ii) activate caspase mediated apoptosis, in cancer cells (e.g., breast cancer cells) that over-express IL-25 receptor compared to non-cancer cells.

The present invention is not limited by the type of cancer or cancer cells that are treated. In certain embodiments, the cancer types that are treated include, but are not limited to, sarcomas and carcinomas such as, but not limited to: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma.

### DESCRIPTION OF THE FIGURES

FIG. 1 shows the 10-50 kDa fraction of the CDMD from differentiating MCF10A cells exerts cytotoxic activity on MCF7 cells in 3-D culture. Morphogenesis of MCF7 cells after (1) 15 h, (2) 4 days and (3) 7 days of growth in 3-D matrix under the treatment with (a) no CDMD, (b) total CDMD, (c) pelleted fraction, (d) total supernatant, (e) 10-50 kDa fraction and (f) >50 kDa fraction.

FIG. 2 shows cell viability assay on MCF7 cells after 7 days of growth in 3-D culture under the same condition as FIG. 1.

FIG. 3 shows fractionated CDMD from differentiating MCF10A cells does not affect the morphogenesis of MCF10A cells in 3-D culture. Morphogenesis of MCF10A cells after (1) 15 h, (2) 4 days and (3) 7 days of growth in 3-D matrix under the treatment with (a) no CDMD (b) total CDMD, (c) pelleted fraction, (d) total supernatant, (e) 10-50 kDa fraction and (f) >50 kDa fraction.

FIG. 4 shows cytotoxic activity in each day fraction of CDMD. Each day fraction of CDMD from differentiating MCF10A cells was collected and fed to MCF7 cells once a day for a week. Percent viable cell number with respect to control cells cultured without CDMD was calculated.

FIG. 5 shows immunoprecipitation of BMP10 and FGF11 from 10-50 kDa fraction of the CDMD.

FIG. 6 shows cell viability assay on MCF7 cells after 7 days of growth in 3-D culture with 10-50 kDa fraction of CDMD immunodepleted of FGF11 and BMP10.

FIG. 7 shows 293 cells stably expressing antithrombin III (ATIII), IL-1F7 or IL-25. a-tubulin (a-TUB) serves as an internal loading control.

FIG. 8 shows cell viability assay on MCF7 cells in 3-D culture after 7 days of treatment with ATIII, IL-1F7 and IL-25 individually or in combination.

FIG. 9 shows cell viability assays on MECs in 3-D culture after 7 days of treatment with IL-25.

FIGS. 10a-e show differentiating mammary acinus structure in 3-D matrix at (a) day 0, (b) day 1, (c) day 2, (d) day 4 and (e) day 6, captured with phase I reflector at 200× magnification. Scale bar: 10 mm. FIG. 10f shows a Western blot analysis on the expression of IL25 by differentiating acini in 3-D culture (b-actin serves as an internal control).

FIG. 11 shows that IL25 exhibits anti-tumor activity both in vitro and in vivo. FIG. 11A shows pooled gel filtrated fractions containing glycosylated IL25 (fraction NO: 21-23) compared to pooled fractions containing other glycosylated proteins (fraction NO: 17-20) analyzed by Coomassie staining (CS, left) and western blot (WB, right). Note the abundant BSA eluted in 17-20 fraction and its carryover in IL25 fraction (21-23 fraction). FIG. 11B shows the number of colonies formed by different breast cell lines (a normal cell line: MCF10A; four breast cancer cell lines: MCF7, MDA-MB468, SKBR3 and T47D) after treatment with IL25 at different concentrations. Error bars: ±SD. FIG. 11C shows the sizes of tumors grown from MDA-MB468 cells xenografted in the mammary fat pads of nude mice after the treatment with vehicle (Ctrl, n=7) or IL25 (300 ng, n=8). p-value=0.0016. Error bars: ±SD. FIG. 11D shows the body weight of mice after the treatment with vehicle (n=7) or IL25 (n=8). Error bars: ±SD. FIG. 11E shows excised tumors after 1 month of treatment. (top) Ctrl tumors; (bottom) IL25-treated tumors. FIG. 11F shows H & E stained sections of (a) control and (b,c) IL25-treated tumors. (c) is a higher magnification image of (b). Control sample shows actively growing tumor cells, whereas in this IL25-treated sample, the tumor is completely regressed and replaced with macrophage aggregates. Magnifications: (a,c) 400×; (b) 100×. Scale bar: 50 mm.

FIG. 12 shows that IL25 receptor (IL25R) is highly expressed in breast tumor cells, but not in normal MECs. FIG. 12A shows RT-PCR analysis for the expression of IL25R in breast cell lines (a-tubulin (a-TUB) serves as an internal control). FIG. 12B shows a Western blot analysis for the expression of IL25R in different breast cell lines (b-actin serves as an internal control). FIG. 12C shows specimens of (a) nontumorous human breast tissue vs. (b) human breast cancer immunostained against IL25R. Membranous staining of IL25R is seen in tumor cells and surrounding inflammatory cells, but not in nonmalignant MECs. The images were captured at 400× magnification. Scale bar: 25 mm FIG. 12D shows survival analysis of patients with IL25R(+) and IL25R(−) tumors.

FIG. 13 shows IL25 induces apoptosis of breast cancer cells through receptor-mediated apoptosis. FIG. 13A shows a Western blot analysis for the cleavages of caspases-8, -3 and PARP in breast cancer cells (MDA-MB468) vs. normal MECs (MCF10A) after treatment with IL25 (500 ng/ml, ˜25 nM) for different periods of time (b-actin serves as an internal control). FIG. 13B shows Western blot analysis showing depletion of IL25R in MDA-MB468 cells after a specific siRNA treatment. Luciferase (Luc) siRNA was used as a nonspecific control. FIG. 13C shows Western blot analysis for the expressions of effector proteins downstream of IL25 signaling in MDA-MB468 cells depleted of IL25R vs. control luciferase. b-actin serves as an internal control. FIG. 13D shows alignment of the C-terminal region of IL25R (aa.362-467, SEQ ID NO:15) with the death domains of FAS receptor (FAS-R, aa.205-293, SEQ ID NO:16) and TNF receptor 1 (TNF-R1, aa.352-441, SEQ ID NO:17) using ClustalW program. The residues within the aligned region are renumbered as indicated (1-106). (*) signs are identical residues; (:) signs are highly similar; (.) signs are similar; and (+) signs are residues highly conserved among death domain-containing proteins (27). FIG. 13E shows co-immunoprecipitation analysis for the interactions of IL25R with death domain adaptor proteins FADD and TRADD. 1/20 of the input protein was shown. b-actin serves as an internal control. FIG. 13F shows a schematic for the cytotoxic activity of IL25 specific to breast cancer cells expressing the receptor IL25R.

FIG. 14 shows that the death domain of IL25R renders cells sensitive to apoptotic signaling of IL25. FIG. 14A shows schematics for IL25R protein expressed: Wt: wild-type full length IL25R protein; ΔTRAF6: mutant with a deletion in TRAF6 binding domain (a.a. Δ339-341); and ΔDD: mutant with a deletion in a death domain (a.a. 4376-387); FIG. 14B shows a Western blot showing IL25R protein (Wt, ΔTRAF6 or ΔDD) ectopically expressed in MCF10A cells compared to that in the parental cells (Ctrl). β-actin serves as an internal control. FIG. 14C shows a Western blot analysis for the cleavages of caspases-3 and PARP in MCF10A cells expressing IL25R protein (Wt, ΔTRAF6 or ΔDD) and in parental cells (Ctrl) after treatment with IL25 (500 ng/ml, ˜25 nM) for different periods of time. β-actin serves as an internal control. FIG. 14D shows a schematic for the cytotoxic activity of IL25 specific to breast cancer cells expressing the receptor IL25R.

FIG. 15A shows the amino acid sequence of human IL-25 (SEQ ID NO:15) and FIG. 15B shows the nucleic acid sequence of human IL-25 (SEQ ID NO: 16).

### DESCRIPTION OF THE INVENTION

The present invention provides methods, kits, and compositions for treating cancer with cytotoxic agents. Preferably, the cytotoxic agents are selected from: IL-25, BMP10, FGF11, VDBP, ATIII and IL1-F7, and any combination thereof. In other preferred embodiments, the cancer is breast cancer. These agents can be supplied, for example, as proteins or as part of nucleic acid expression vector (e.g., an adeno-virus encoding one of the cytotoxic agents).

Proliferation and differentiation are coordinated in a way that activation of differentiation in normal cells is typically associated with cessation of proliferation. Therefore, a balance between the two is usually disrupted in tumor cells. Based on this premise, a differentiation-inducing therapy, focused on suppressing erratic proliferation of tumor cells by reactivating differentiation, is one of the tumor dormancy therapies proposed by Uhr et al (Uhr et al., 1997). At present, the most successful differentiation-inducing therapy is the application of all-trans-retinoic-acid (ATRA) to acute promyelocytic leukemia (Castaigne et al., 1990; Huang et al., 1988; Warrell et al., 1991). However, utilization of tumor dormancy therapy in solid tumors is underdeveloped and awaits an innovation.

During the development of the present invention, it was observed that normal differentiating MECs (mammary epithelial cells) secrete factors that can induce differentiation of breast cancer cells. More importantly, a subset of these factors, which were enriched in the 10-50 kDa fraction of CDMD from differentiating normal MECs, exerts cell killing activity on breast cancer cells without affecting normal MECs (See, Example 1). Utilization of such natural factors that specifically suppress proliferation and induce cell death of breast cancer cells will serve as a novel tumor dormancy therapy for treating breast cancer.

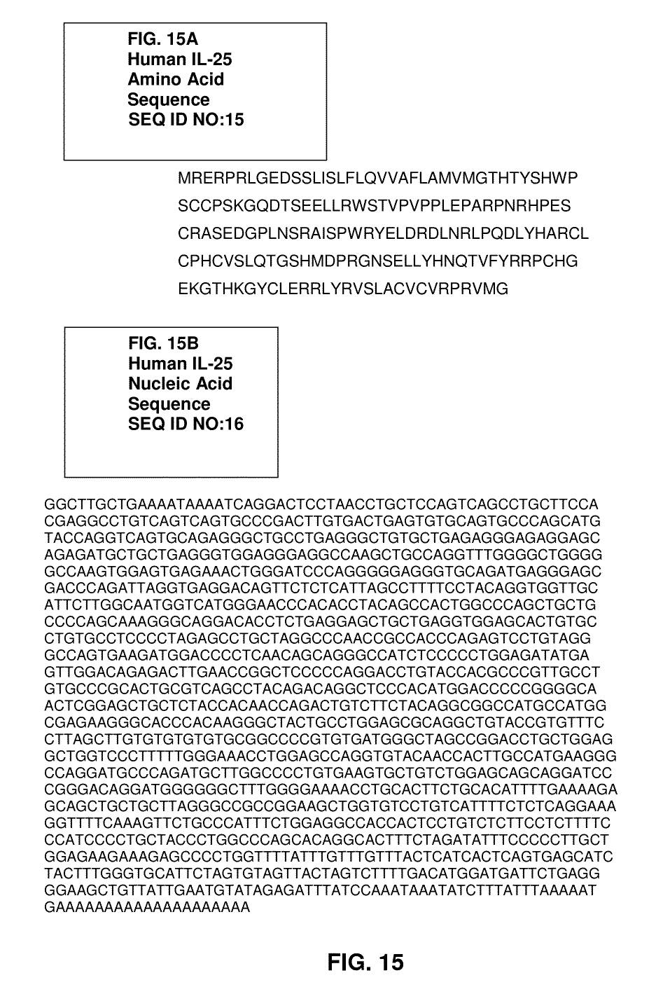

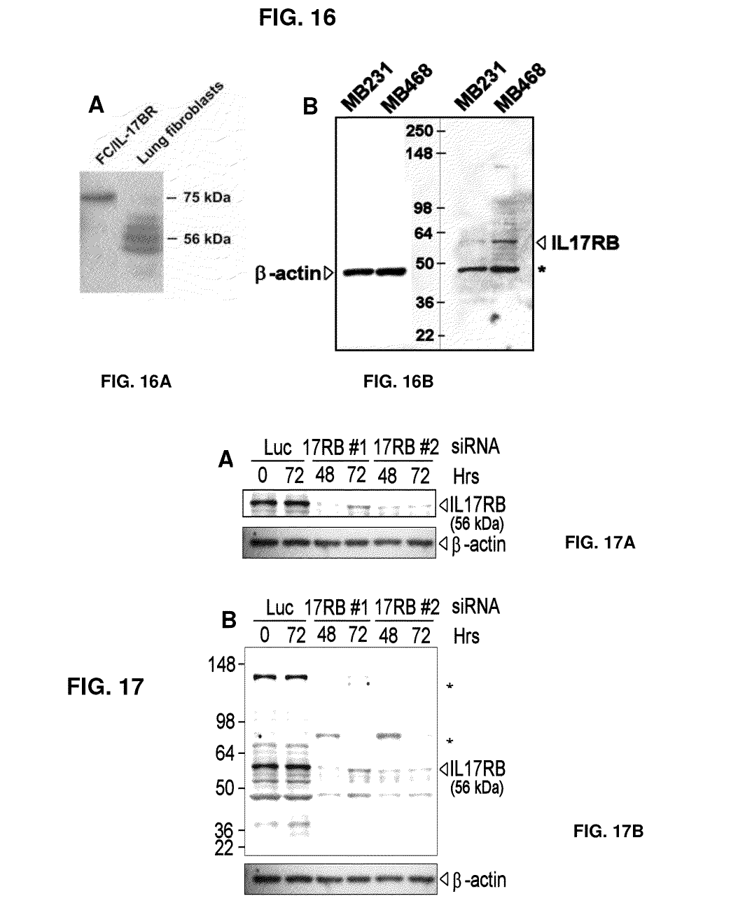

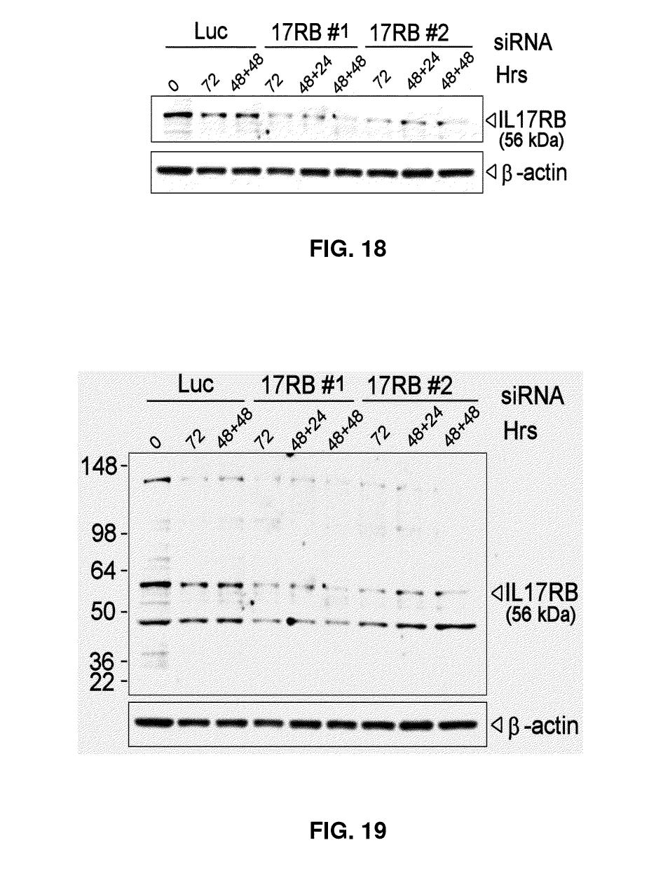

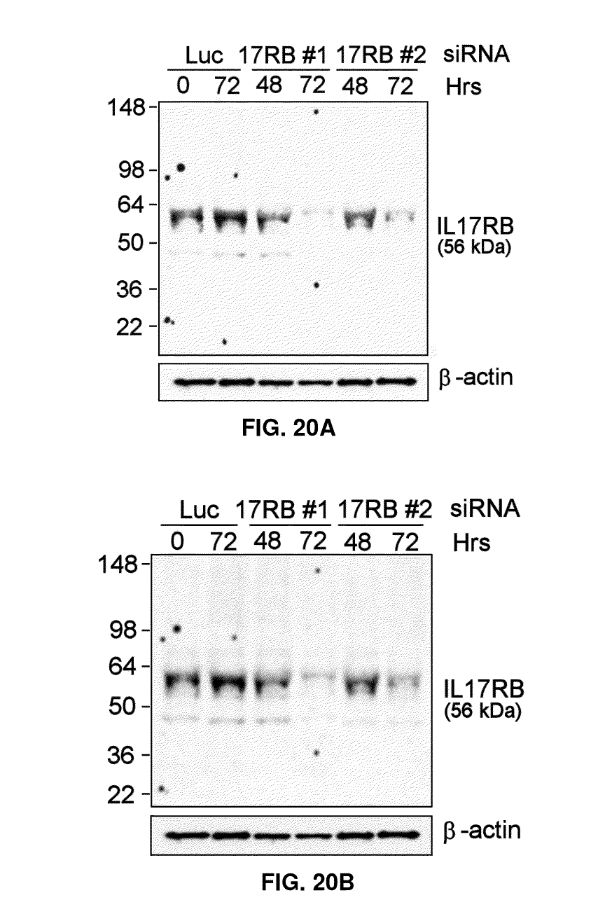

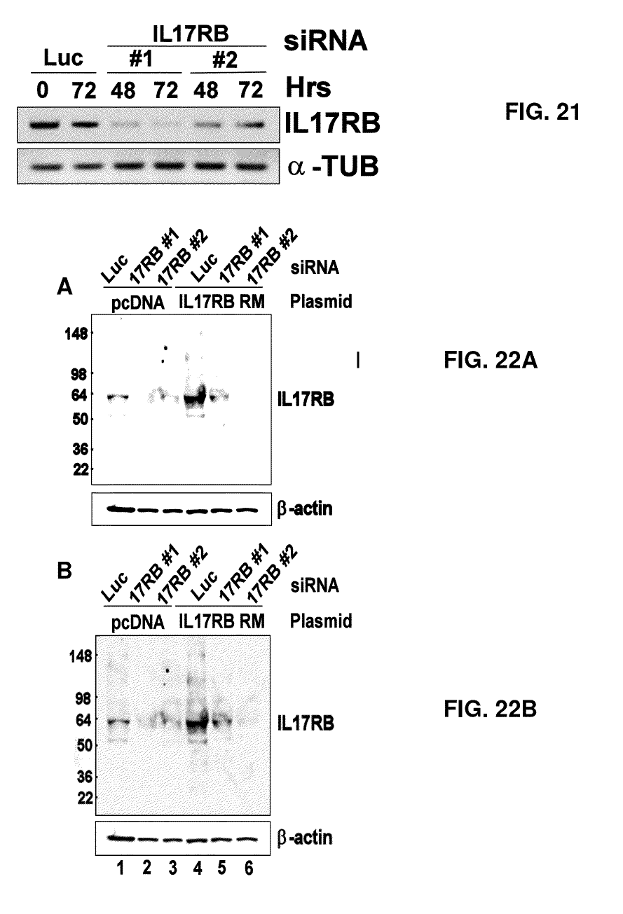

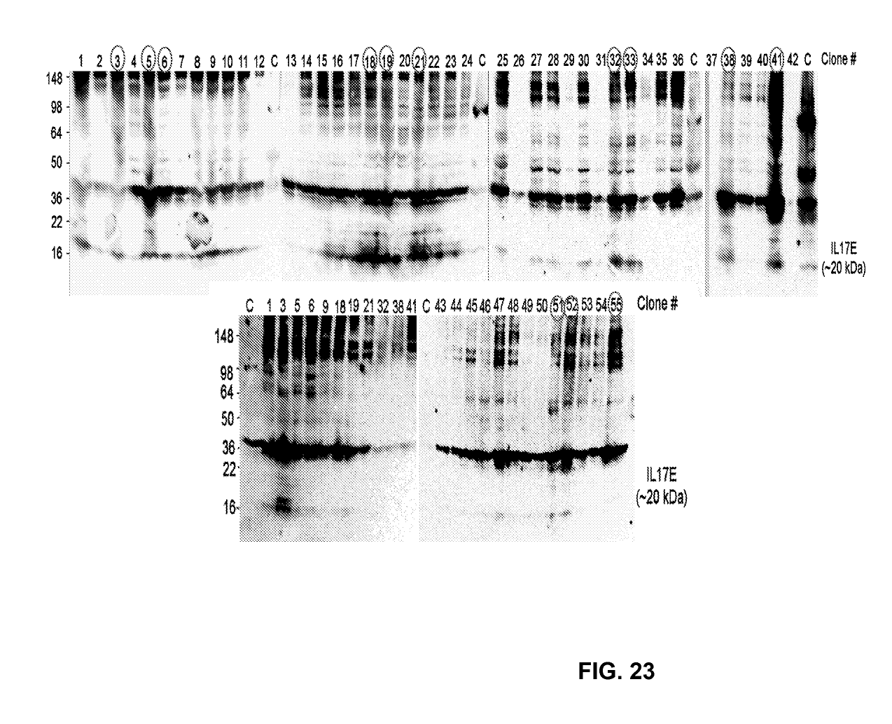

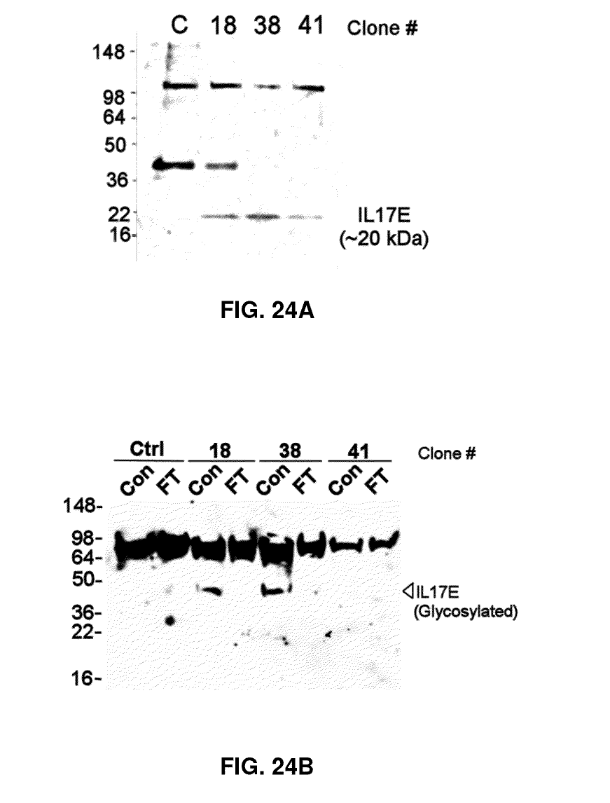

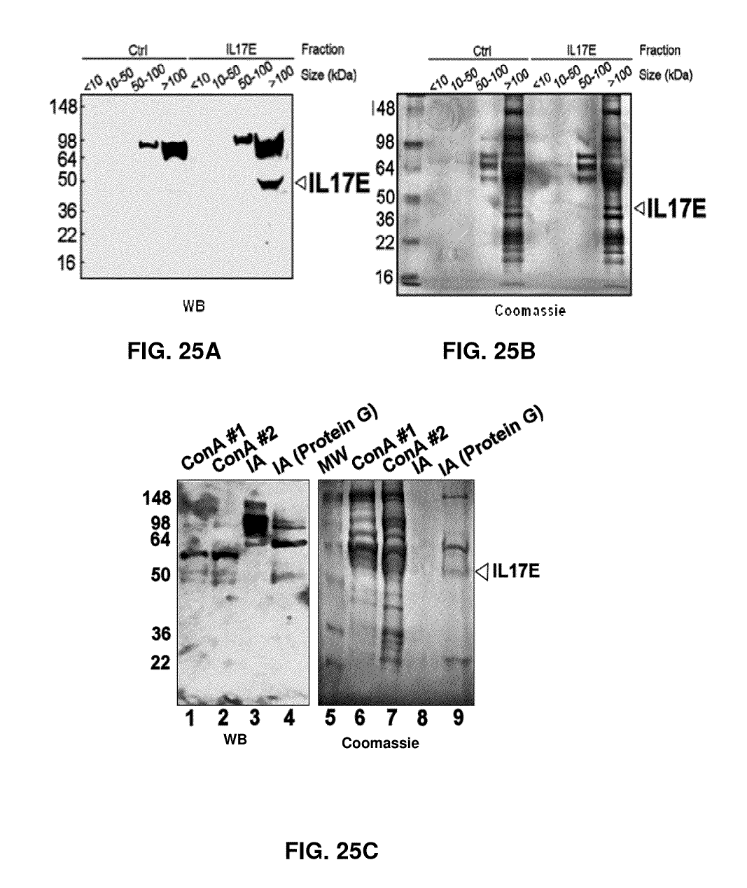

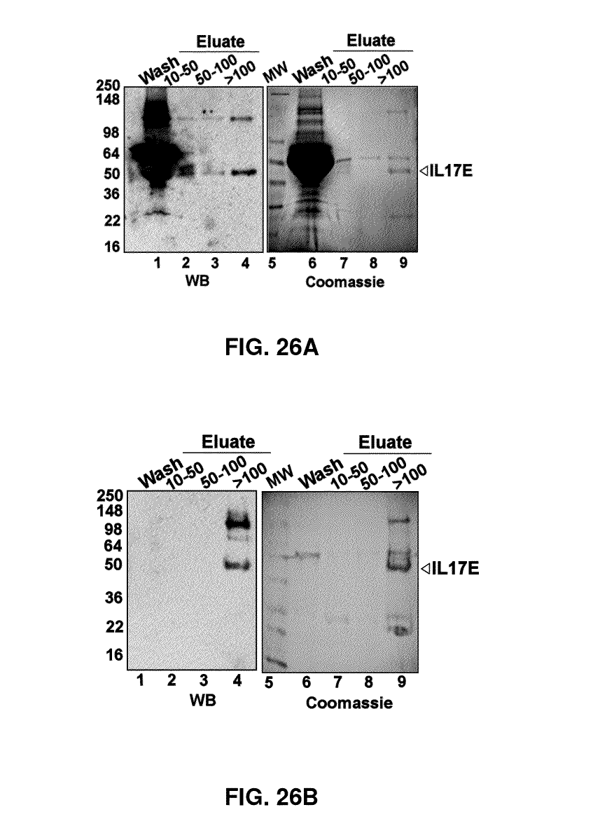

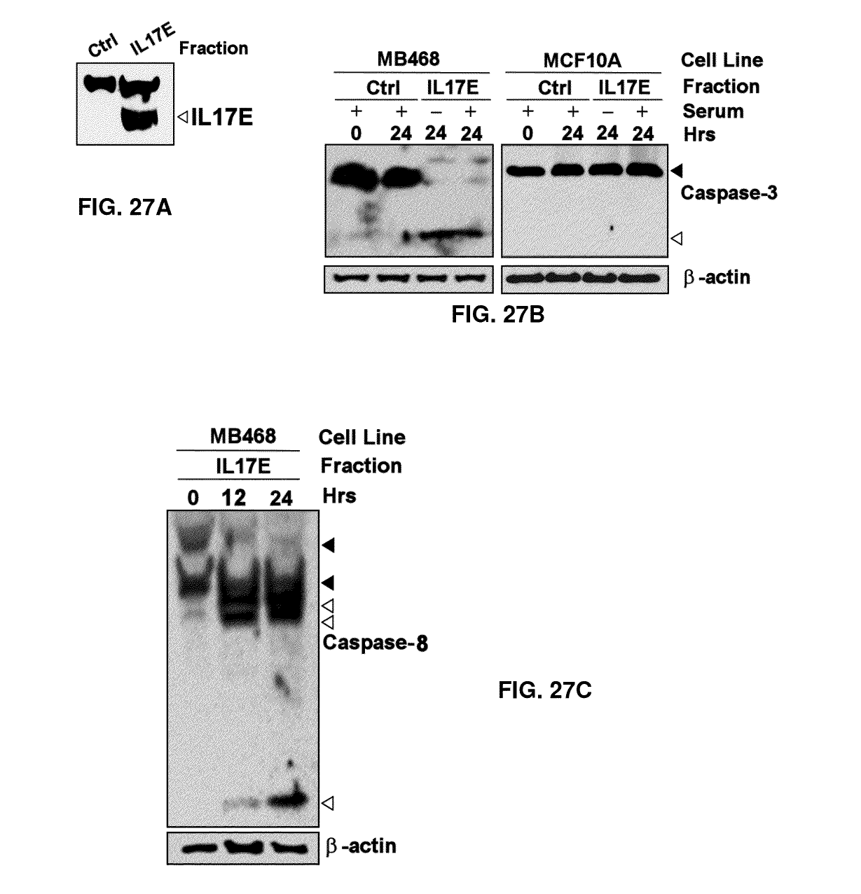

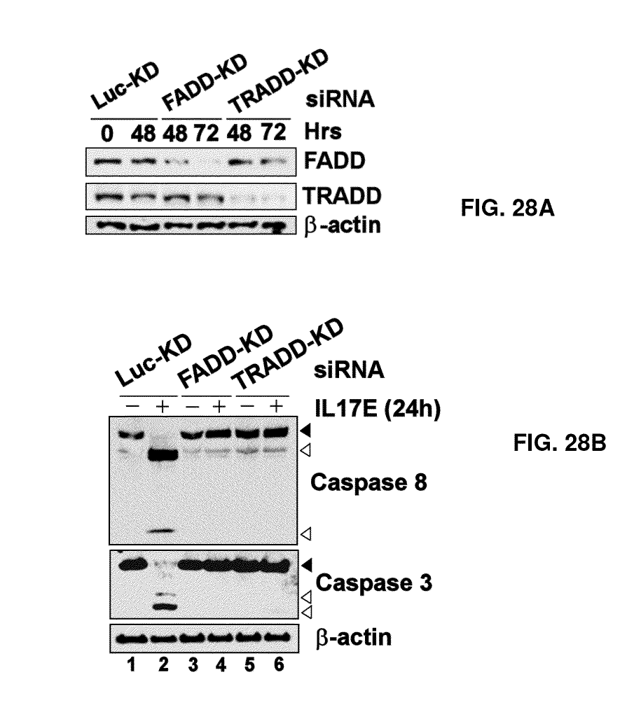

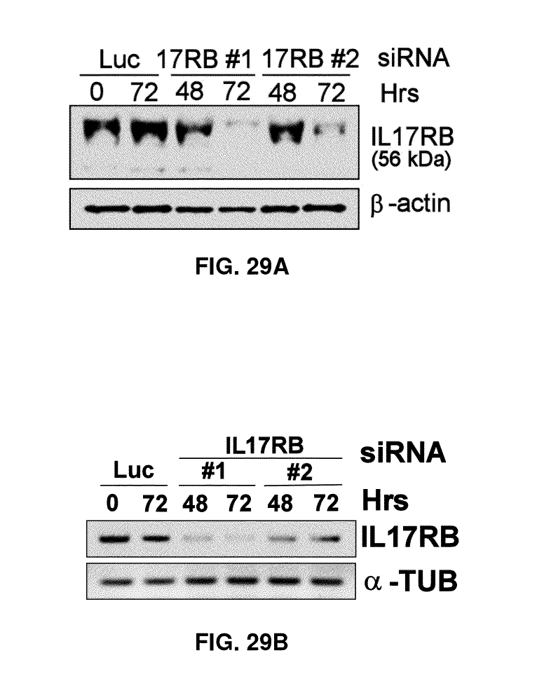

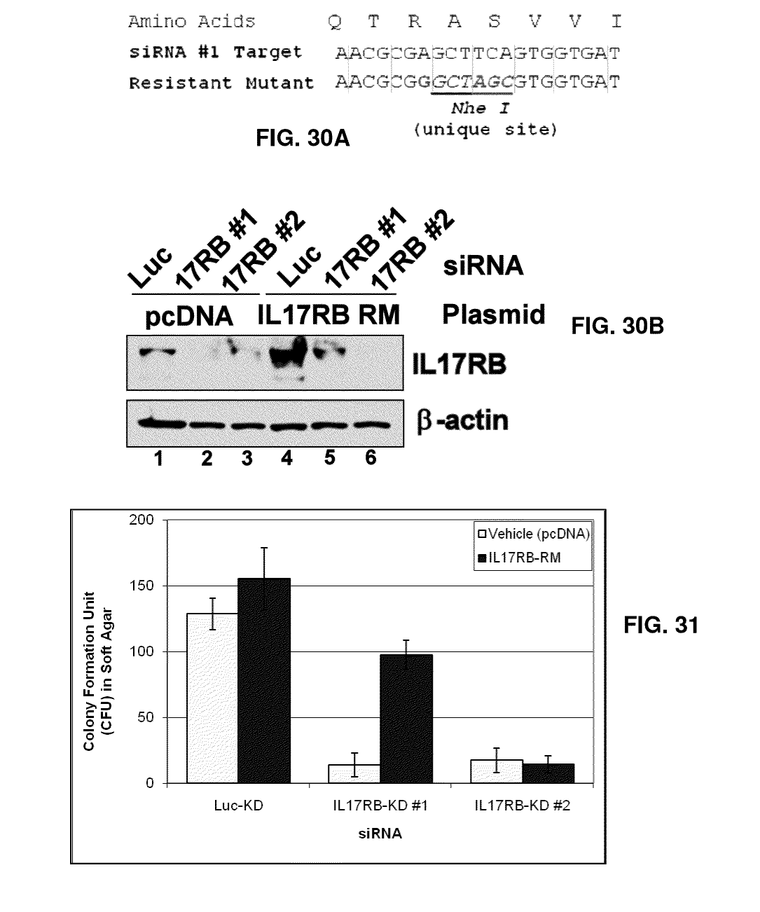

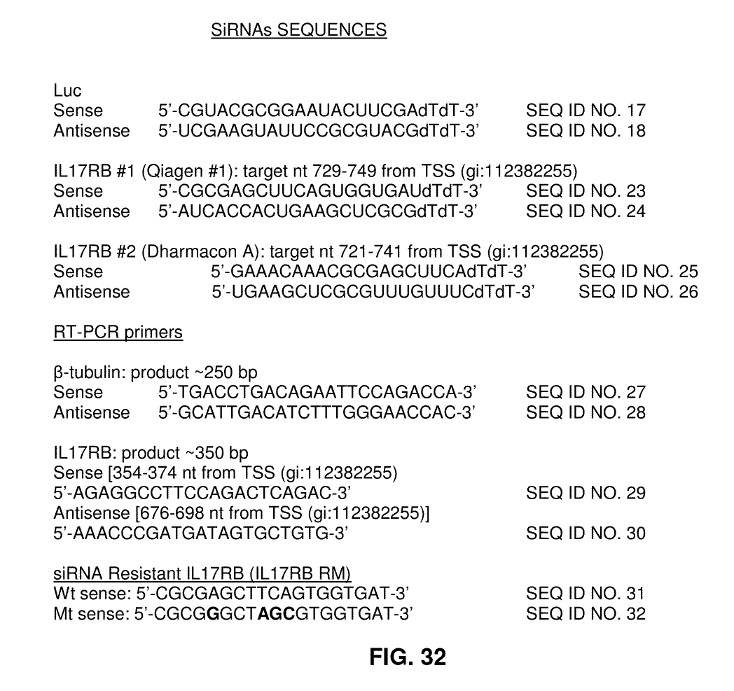

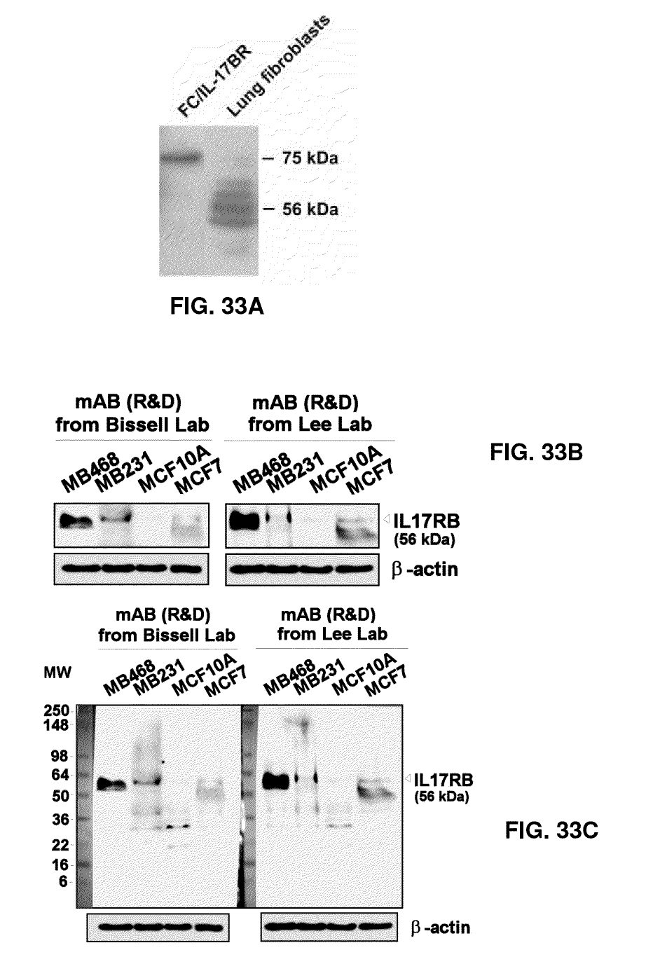

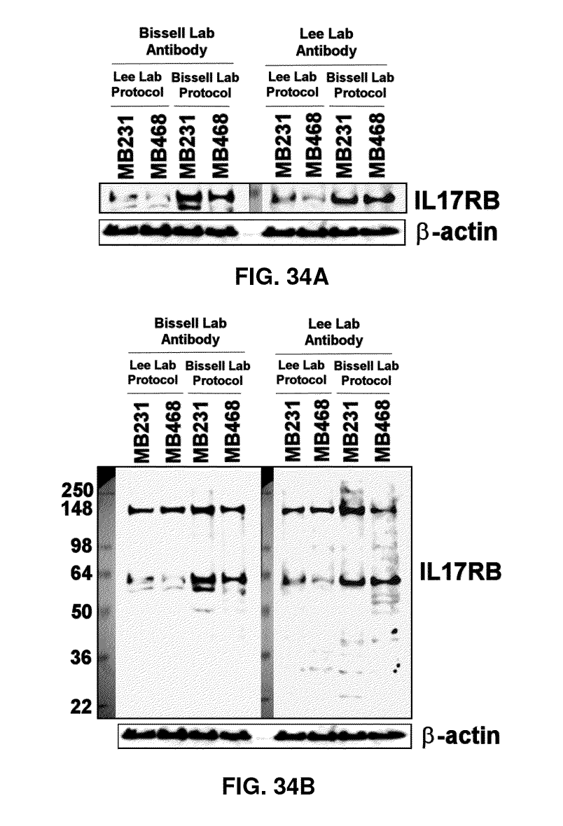

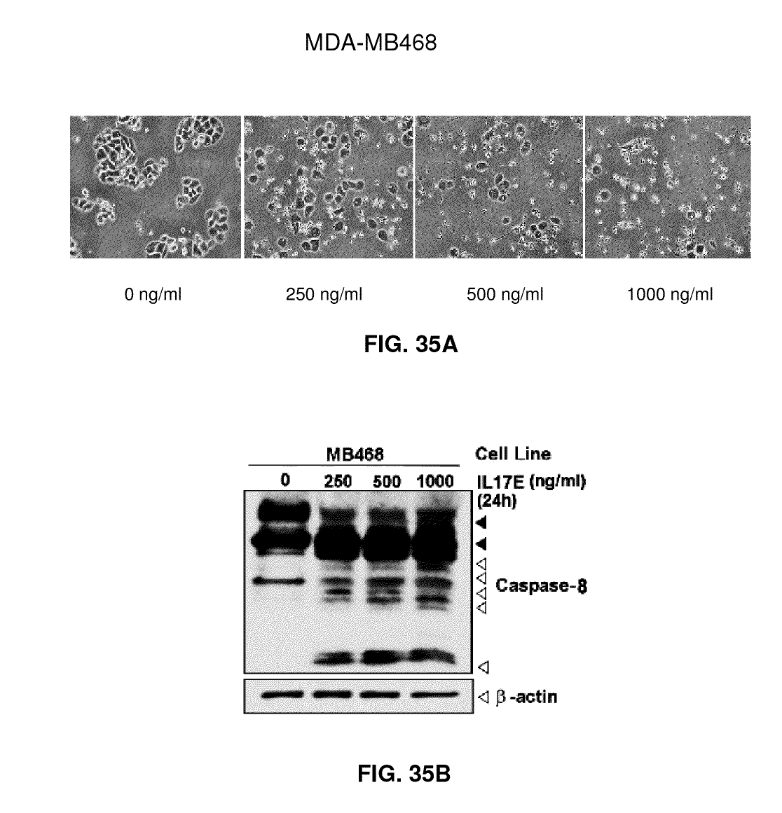

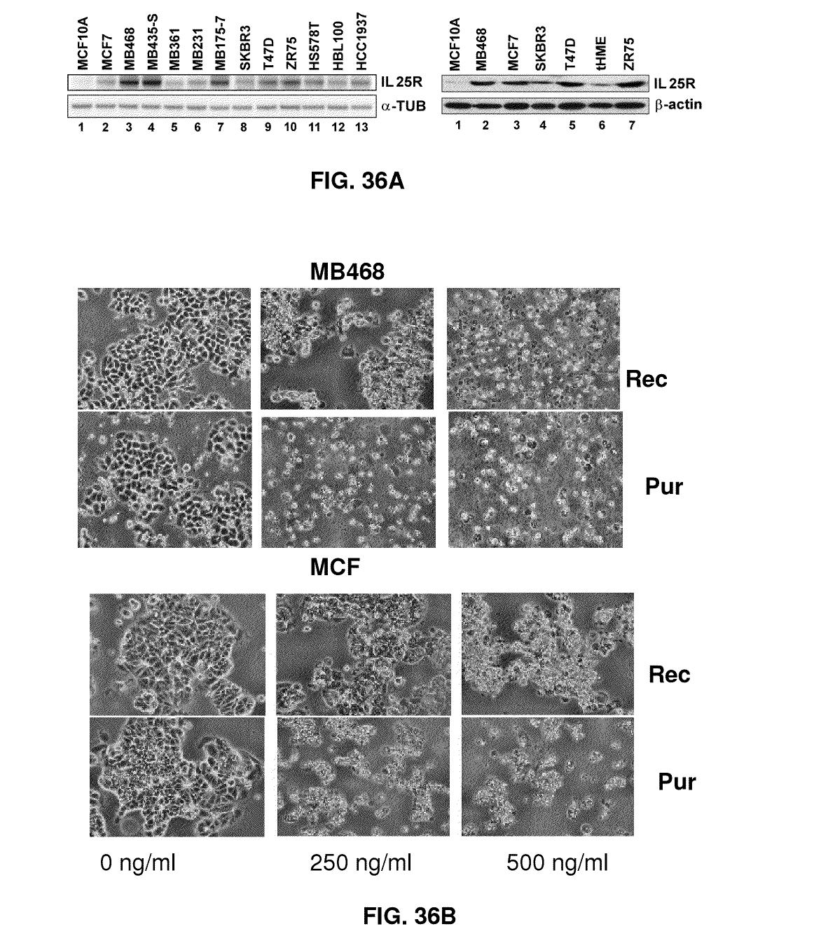

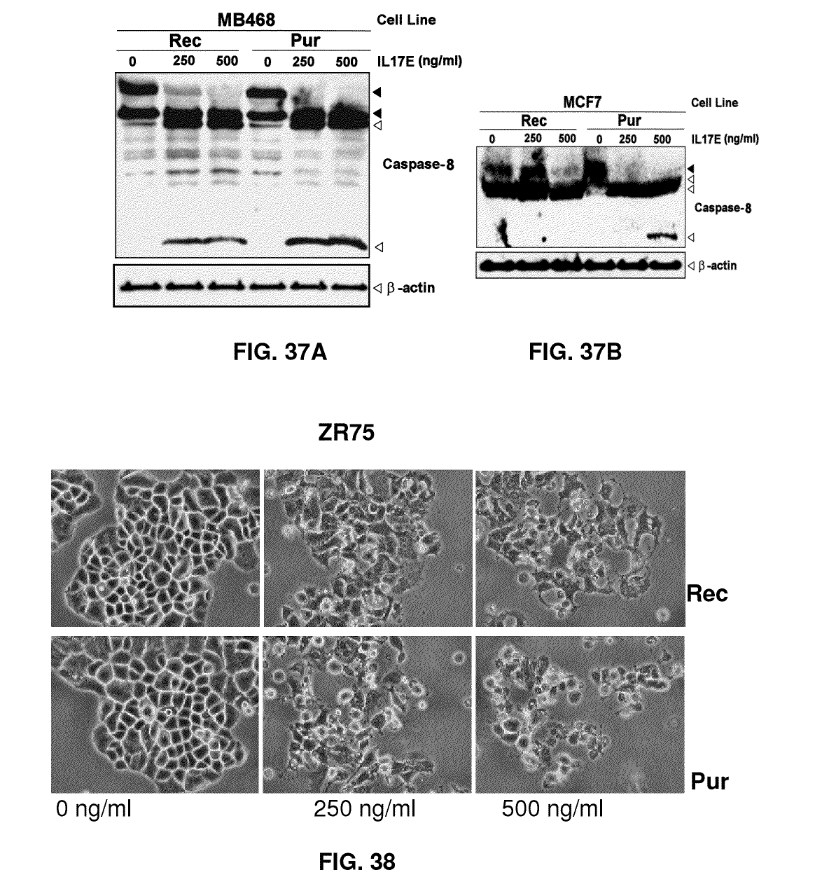

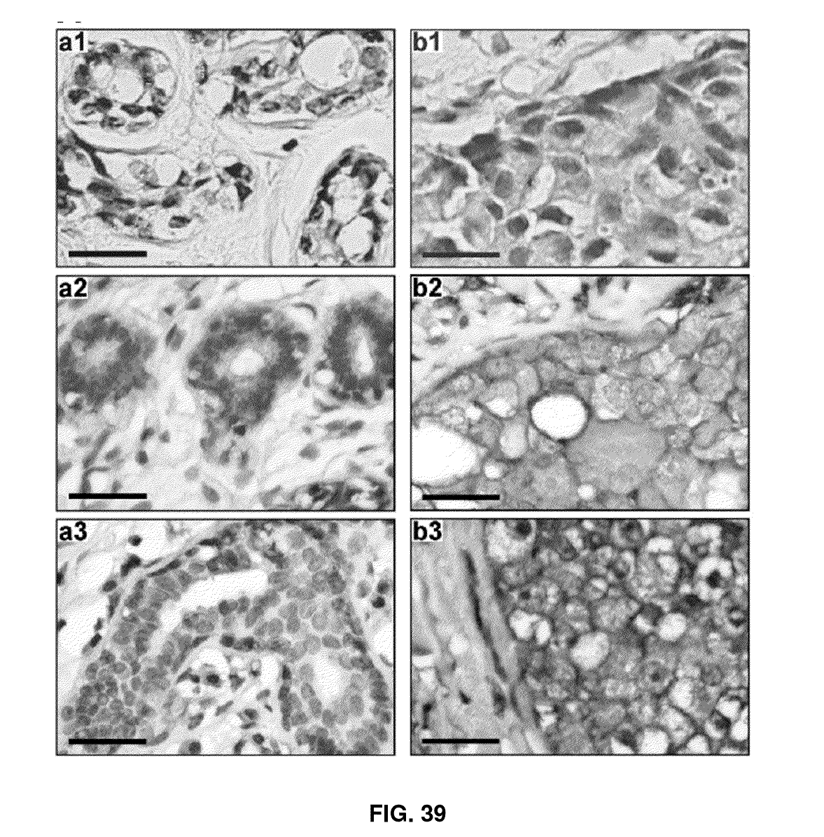

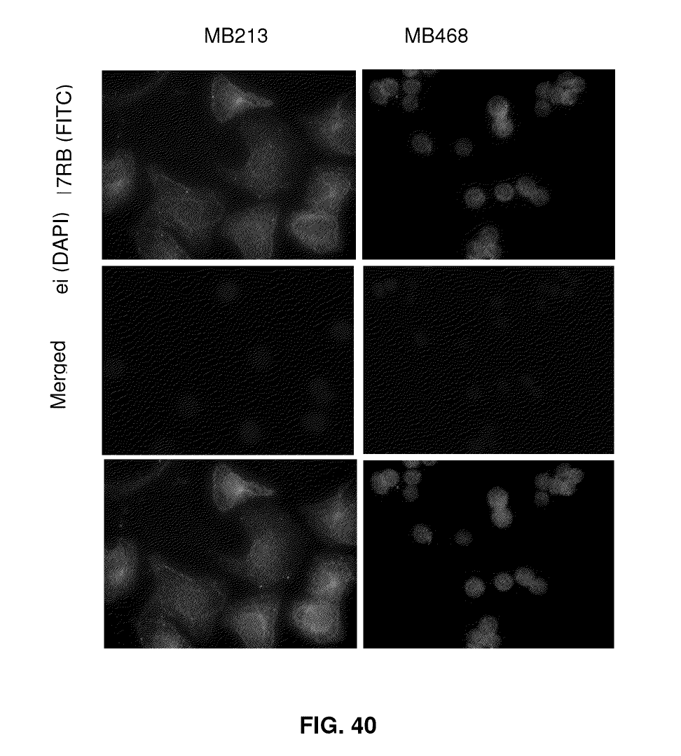

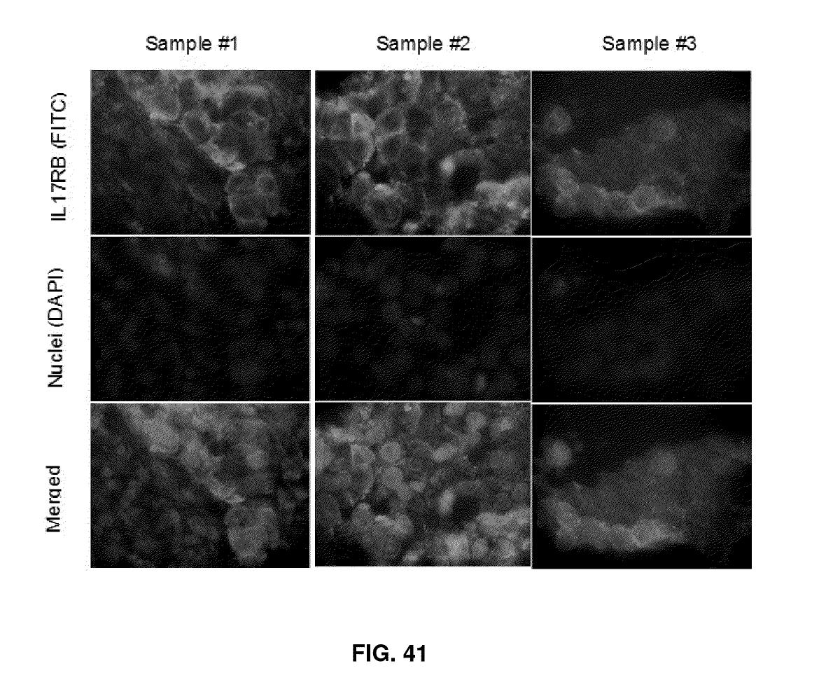

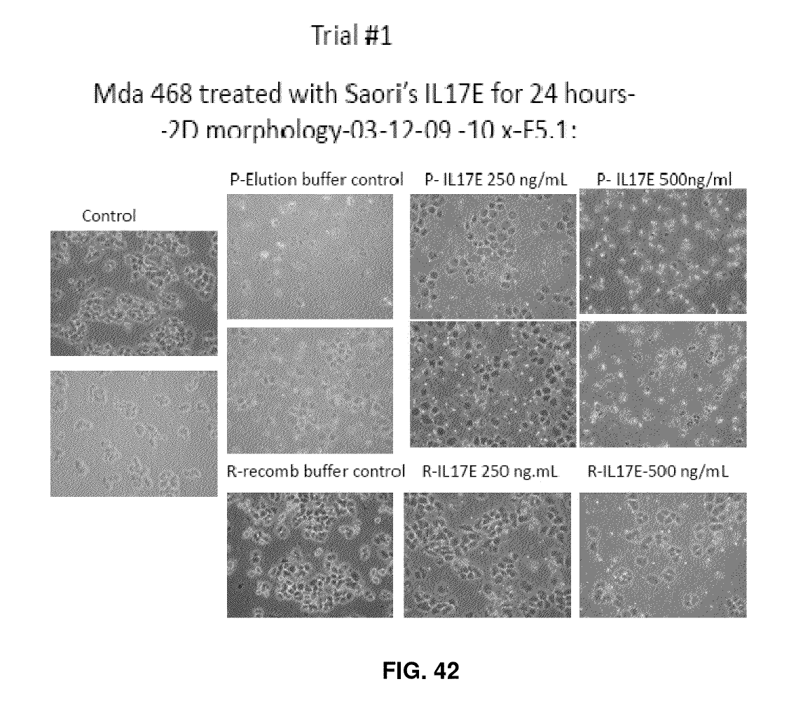

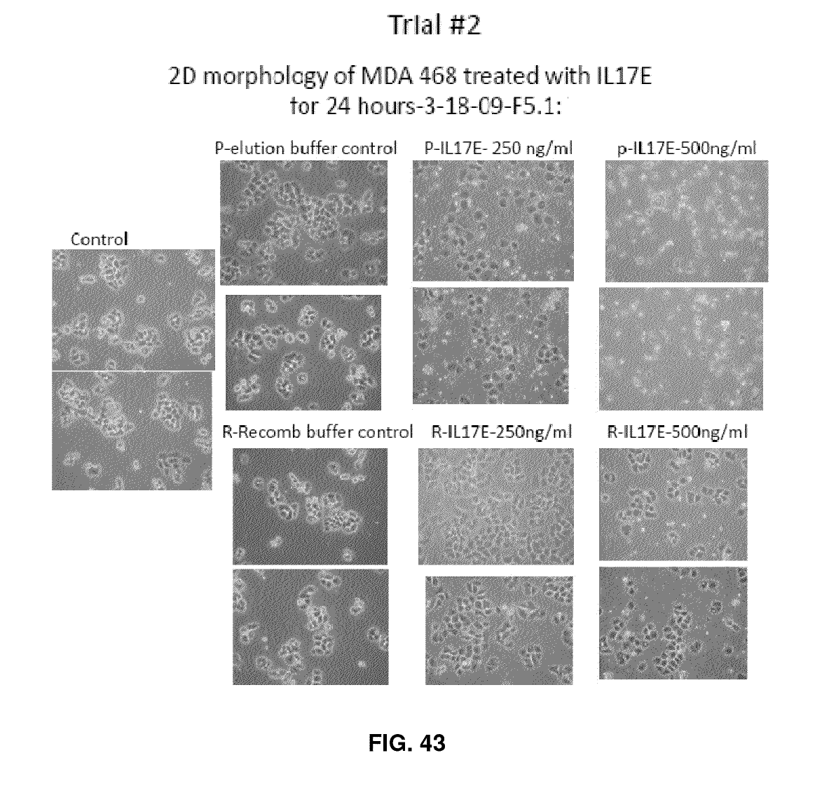

In certain embodiments, the cytotoxic agent used for treating cancer is an agent configured to bind an IL-25 receptor. In preferred embodiments, the agent is configured to bind an IL-25 receptor and activate caspase mediated apoptosis. Such agents include IL-25 (e.g, human IL-25, see FIG. 15), IL-25 variants (including truncations, deletions, and substitutions), anti-IL-25R antibodies or antibody fragments (e.g., Fab) and mimetics (e.g., small molecules that bind to IL-25R). It is known that IL-25 binds the IL-25R and therefore can serve as the cytotoxic agent. Additional agents can be identified by screening candidate IL-25R binding agents in various screens. For example, one of skill in the art could generate an IL-25 variant by using part of the nucleic acid sequence shown in FIG. 15B. Such a candidate molecule could be screened in an IL-25R binding assay. Any type of suitable binding assay known in the art can be used (e.g., binding assay where IL-25R proteins and the candidate agent are both labeled, or one is labeled and one is attached to a solid support, may be employed). Cell based assays may also be employed. A preferred cell based assay would be similar to the present Examples (below) where IL-25 is substituted for a candidate agent to see if the candidate agent will bind IL-25R and cause cell death. In vivo type assays may also be employed. A preferred in vivo based assay is as shown in the Examples below, where IL-25 is replaced with the candidate agent to see if the candidate agent will reduce or eliminate tumors in an animal model.

### EXAMPLES

The following examples are provided in order to demonstrate and further illustrate certain preferred embodiments and aspects of the present invention and are not to be construed as limiting the scope thereof.

### Experimental Procedures

The following experimental procedures were used for the examples below.

Cell Cultures

Human mammary epithelial MCF10A cells were cultured as described (Debnath and Muthuswamy); human breast cancer cell lines (MCF7, MDA-MB361, T47D, ZR75, MDA-MB468, MDA-MB435-S, MDA-MB231, MDA-MD175-7, SKBR3, HS578T, HBL100 and HCC1937) and human embryonic kidney cells 293T were cultured as described (Furuta et al).

Assessment of the Cytotoxicity of CDMD from MECs

MCF10A cells were plated at 4×10⁴ cells in a 35 mm-dish coated with 1 ml Growth Factor Reduced Matrigel (BD Biosciences) and covered with 3 ml growth medium supplemented with 2% Matrigel as described (Debnath and Muthuswamy). After 15 hrs CDMD (2.5 ml) was collected and separated into soluble and pelleted fractions by centrifugation at 14,000 for 30 min. The soluble fraction was size-fractionated with Centricon-10 and -50 units (Millipore) following the manufacturer's instruction; the pellet was resuspended in 400 ml of growth medium. 400 ml each of the following fractions were obtained: 1) total CDMD, 2) pellet, 3) total supernatant, soluble fractions of 4) >50 kDa, 5) 10-50 kDa and 6) <10 kDa. All the fractions were reconstituted with the essential growth factors and 2% Matrigel and applied to MCF7 or MCF10A cells seeded at 5000 cells/well in Matrigel-coated 8-well chamber slides. The collection/application of CDMD was performed every 12-15 hrs for 1 week. To determine when differentiating MECs produce cytotoxic factors, the 10-50 kDa fraction of CDMD was harvested at each day (days 0-6) from differentiating MCF10A in 3-D matrix using the above-mentioned condition. Different day fractions were individually applied to MCF7 cells plated at 1000 cells/well in Matrigel-coated 96-well plate. Fresh CDMD was applied every 24 hours. For cell number counting, cells were recovered from Matrigel after 1 week by digestion with dispase (BD Biosciences), and the viable cell numbers were measured using trypan blue exclusion method.

Microscopic Imaging

Microscopic imaging of live cells was performed on a Zeiss Axiovert 200 M equipped with Hamamatsu Photonics K.K. Deep Cooled Digital Camera using Axiovision 4.5 software (Carl Zeiss). The images were captured with phase I at 100× or phase II reflector at 200× magnification. Photomicrographs of histology specimens were taken with Zeiss Axioplan 2 Imaging platform and AxioVision 4.4 Software at 100× or 400× magnification.

Mass Spectrometric Analysis

The cytotoxic fraction (10-50 kDa, day 4) of CDMD from differentiating MCF10A cells and the soluble fraction of CDMD from MCF7 cells in 3-D matrix (day 4) were collected and analyzed for mass spectrometry as described (Wang et al., and Chalkley et al.).

Sample Preparation for Mass Spectrometric Analysis

The cytotoxic fraction (10-50 kDa, day 4) of CDMD from differentiating MCF10A cells and the soluble fraction of CDMD from MCF7 cells in 3-D matrix (day 4) were collected. Proteins in each medium were concentrated by trichloroacetic acid precipitation and dissolved in boiling SDS sample buffer. Proteins were resolved by SDS-PAGE (10%) and visualized with Coomassie Blue staining Gel slices (2 mm thickness) were excised, destained with 25 mM NH₄HCO₃ in 50% MeOH and digested with 50 ng/ml trypsin in 50 mM NH₄HCO₃ for 24 h at 37° C. Peptides were extracted from gel slices with 3 volume of 50% acetonitrile, vacuum-dried and resuspended in 0.1% formic acid. Following sample clean up in C18 ZipTips (Millipore), peptides were eluted with 0.1% formic acid in 50% acetonitrile.

Liquid Chromatography and Tandem Mass Spectrometry (LC-MS/MS)

For LC-MS/MS analysis, the digests were first separated by cation exchange chromatography (polysulfoethyl A column, Nest Group) using a linear gradient between solvents A (5 mM KH2PO4, 30% acetonitrile, pH 3) and B (solvent A with 350 mM KCl) at a flow rate of 0.2 ml/min. Fractions were collected on the basis of UV absorbance (215 and 280 nm) and desalted with C18 micro spin columns (Vivascience). LC-MS/MS was carried out by nanoflow reverse phase liquid chromatography (RPLC) (Ultimate LC Packings) coupled on-line to QSTAR XL tandem mass spectrometer (Applied Biosystems). RPLC was performed using a capillary column (75 μm×150 mm) packed with Polaris C18-A resin (Varian Inc.), and the peptides were eluted using a linear gradient between solvents A (2% acetonitrile, 0.1% formic acid) and B (98% acetonitrile, 0.1% formic acid) at a flow rate of 250 nl/min. Each full MS scan was followed by three MS/MS scans where three most abundant peptide ions were selected to generate tandem mass spectra. Two LC-MS/MS runs were performed on the same sample to improve the dynamic range of mass spectrometric analysis.

Protein identification

For MS data analysis, monoisotopic masses (m/z) of peptide ions were obtained from the tandem mass spectra using Mascot script in Analyst QS version 1.1 software (Applied Biosystems) with the mass accuracy of ±200ppm. Certain chemical modifications (i.e., N-terminal acetylation or pyroglutamine, methionine oxidation, asparagine deamination, carbamylation of the N-terminus and lysine, phosphorylations of serine, threonine and tyrosine) were selected as variables during the peptide search using Batch-Tag program in Protein Prospector version 4.25.0 software (UCSF). Both Uniprot and NCBInr public databases were queried to identify the proteins. Search Compare program in Protein Prospector was used to summarize the results including protein scores and discriminating score among peptide fragments. The result obtained for CDMD from differentiating MCF10A cells was compared to that for CDMD from MCF7 cells in 3-D culture, and proteins only present in the former sample were identified. Proteins with the best score >20 and discriminating score <6 were considered significant.

Immunodepletion

The cytotoxic fraction (10-50 kDa, days 3-5) of CDMD was harvested from differentiating MCF10A cells in 3-D culture. The medium was divided into six fractions (2 ml each), and each fraction was clarified with 100 ml of protein G sepharose beads at 4° C. for 2 hours. One mg of antibody against BMP10, FGF11, ATIII, IL1F7, IL25, VBP or p84 (Ctrl) was added to each fraction and incubated at 4° C. overnight. Antibody-protein complex was precipitated by 100 ml protein A/G sepharose beads (1:1) at 4° C. for 2 hrs. The immunoprecipitates were washed in TEN buffer (10 mM Tris-HCl (pH8.0), 0.25 mM EDTA, 50 mM NaCl) supplemented with 0.1% NP-40 and protease inhibitors, then analyzed by western blot. 1/20 of the immunodepleted fraction was also examined by western analysis, and depletion was repeated 3 times to ensure complete loss of a target protein. Depleted fractions were reconstituted with the essential growth factors and 2% Matrigel, then used to plate MCF7 cells seeded at 5000 cells/well in Matrigel-coated 8-well chamber slides. The fraction was applied every 24 hours for 1 week. Cells were recovered from Matrigel, and the viable cell numbers were measured.

Stable Cell Lines for ATIII, IL1F7, IL25 and VBP

Full-length cDNA clones of ATIII, IL1F7, VBP (pDR-LIB) and IL25 (pPCR-Script/Amp) were obtained from ATCC. ATIII, IL1F7 and VBP cDNAs were excised at SmaI/XhoI sites and subcloned into EcoRV/XhoI sites of pcDNA3.1/Hyg vector (Invitrogen), while IL25 cDNA was excised at HindIII/NotI and subcloned into pcDNA3.1/Hyg. 293T cells were transfected with the respective plasmid and selected with 70 mg/ml Hygromycin B (Roche). Expression of each protein was confirmed by RT-PCR using primers shown in Table 1. For determining the cytotoxic activity of each factor, 7 ml of CDMD from 293T cells (4×106) was harvested, concentrated by 2 fold with Centricon-10, supplemented with growth factors plus 2% Matrigel and applied to MCF7 cells seeded at 5000 cells/well in Matrigel-coated 8-well chamber slides. Fresh CDMD was applied every 24 hours for one week, and the viable cell numbers were counted. To generate a stable cell line for IL25 purification, IL25 cDNA was subcloned into BamHI site of pQCXIH retroviral vector (BD Biosciences). IL25 retrovirus was generated to establish a stable 293T cell line as described (Furuta et al).

TABLE 1

| | | |
|---|---|---|
| Name | F/R | Sequence |

RT-PCR Primers

| | | |
|---|---|---|
| ATIII | F | 5′-GCT TTT GCT ATG ACC AAG CTG-3′ SEQ ID NO: 1 |
| RT | R | 5′-TGC TTC ACT GCC TTC TTC ATT-3′ SEQ ID NO: 2 |
| IL1F7 | F | 5′-AAA CCC GAA GAA ATT CAG CAT-3′ SEQ ID NO: 3 |
| RT | R | 5′-CCC ACC TGA GCC CTA TAA AAG-3′ SEQ ID NO: 4 |
| IL25 | F | 5′-TTC CTA CAG GTG GTT GCA TTC-3′ SEQ ID NO: 5 |
| RT | R | 5′-CGC CTG TAG AAG ACA GTC TGG-3′ SEQ ID NO: 6 |
| VBP RT | F | 5′-AAT CAA GGC TCA GCA ATC TCA-3′ SEQ ID NO: 7 |
| | R | 5′-CAT CTT TGT TTG TGG GCA ACT-3′ SEQ ID NO: 8 |
| IL25R | F | 5′-AGA GGC CTT CCA GAC TCA GAC-3′ SEQ ID NO: 9 |
| RT | R | 5′-AAA CCC GAT GAT AGT GCT GTG-3′ SEQ ID NO: 10 |
| α- | F | 5′-TGA CCT GAC AGA ATT CCA GAC CA-3′ SEQ ID NO: 11 |
| tubulin | R | 5′GCA TTG ACA TCT TTG GGA ACC AC-3′ SEQ ID NO: 12 |

siRNA

| | | |
|---|---|---|
| IL25R | F | 5′-r(CGC GAG CUU CAG UGG UGA U)dTdT-3′ |
| | | SEQ ID NO: 13 |
| | R | 5′-r(AUC ACC ACU GAA GCU CGC G)dTdT-3′ |
| | | SEQ ID NO: 14 |

Purification of Secreted IL25

CDMD was collected from a stable 293T cell line expressing IL25, supplemented with protease inhibitors and loaded onto a column packed with concanavalin A-sepharose beads (CalBiochem) pre-equilibrated with column buffer (10 mM Tris (pH7.5), 150 mM NaCl, 1 mM CaCl₂, 1 mM MnCl₂). The column was washed with column buffer, and bound glycosylated proteins were eluted with 0.5M a-methyl mannose in column buffer. The eluates were pooled, concentrated with Centricon-10 and then separated by Superdex 200 gel filtration column (HR 10/30, 24mL; Amersham Pharmacia) using elution buffer (50 mM Na₂HPO₄ (pH7.5), 50 mM NaCl) at a flow rate of 0.4 ml/min. Fractions were collected based on UV absorbance at 280 nm and resolved on 10% SDS-PAGE for western analysis.

Colony Formation Assay

Breast cancer cells (MCF7, MDA-MB468, SKBR3 and T47D) at 1000/well and MCF10A cells at 500/well were seeded in 6-well plates in triplicate and maintained for 24 hours. Designated amounts of IL25 were diluted in elution buffer to 100 ml and then in 900 ml growth medium to culture cells. After 10 days cells were stained with 2% Methylene Blue in 50% EtOH, and the numbers of colonies were counted.

Tumor Inhibition Assay in Nude Mice

Animal experiments were performed under federal guidelines and approved by Institutional Animal Care and Use Committee at UCI. Exponentially growing MDA-MB-468 cells at 10×10⁶ in 100 ml of DMEM plus 5% Matrigel were injected into the mammary fat pad of 6-8 wk old athymic female BALB/c-nude mice (nu/nu) (Charles River Laboratories). After tumors grew to 80 mm³ (day 10), mice were randomized into control (n=7) and experimental groups (n=8) to receive on site injection of vehicle (elution buffer, 50 ml) or IL25 (300 ng, 50 ml) once every day for 31 days. Mouse body weights and tumor volumes were measured twice weekly during the course of treatment. Student's t test was used to determine p-value as indicated in the figure legend. At the end of experiments, mice were sacrificed and subjected to pathological examinations. For the safety test, IL25 (1.5 mg) was injected into the tail veins of female C57 mice (vehicle: n=3; IL25: n=5), and signs for systemic stress (e.g., lethargy and weight loss) were monitored daily.

Histology and Immunohistochemistry

Dissected tumors were fixed in 4% paraformaldehyde overnight and embedded in paraffin with a tissue processor. 4-5 mm sections were deparaffinized and hydrated. Tumor xenografts were stained with hematoxylin and eosin, while human breast tumor specimens were processed for immunohistochemistry. Antigen retrieval was performed in 0.01 M citric buffer at 100° C. for 10 minutes. After blocking with 3% H₂O₂ and nonimmune horse serum, the slides were allowed to react with a monoclonal antibody against human IL25R (GeneTex; 1:100 dilution) at 4° C. overnight. The slides were incubated with link antibodies, followed by peroxidase conjugated streptavidin complex (LSAB kit, DAKO Corp.) The peroxidase activity was visualized with diaminobenzidine tetrahydroxychloride solution (DAB, DAKO) as the substrate. The sections were lightly counterstained with hematoxylin. The survival curve of patients was obtained by Kaplan-Meier analysis using XLSTAT-Life Version 2007.4 software.

IL25R siRNA

MB468 cells were plated at 3.5×105/60 mm dish and maintained for 24 hours. Cells were transfected with 400 pmol of annealed IL25R siRNA (Table 1, Qiagen) using Oligofectamine (Invitrogen) according to manufacturer's instruction.

Immunoprecipitation

Nine mg of whole cell lysates were collected in 3 ml of Triton lysis buffer (25 mM Tris (pH7.6), 150 mM NaCl, 1% TritonX-100) supplemented with protease and phosphatase inhibitors. The lysate was divided into three fractions (3 mg protein/1 ml each), and each fraction was clarified with 50 ml of protein G sepharose beads at 4° C. for 2 hours. Two mg of antibody against p84 (Ctrl), IL25R or FADD (Cell Signaling) was added to each fraction and incubated at 4° C. overnight. Antibody-protein complex was precipitated by 50 ml protein A/G sepharose beads at 4° C. for 2 hrs and washed in TEN buffer with 0.1% NP-40 and protease inhibitors. Immunoprecipitates were resolved on 12% SDS-PAGE and detected by western analysis.

### Example 1

### Factors Secreted from Differentiating MEC Kill Breast Cancer Cells

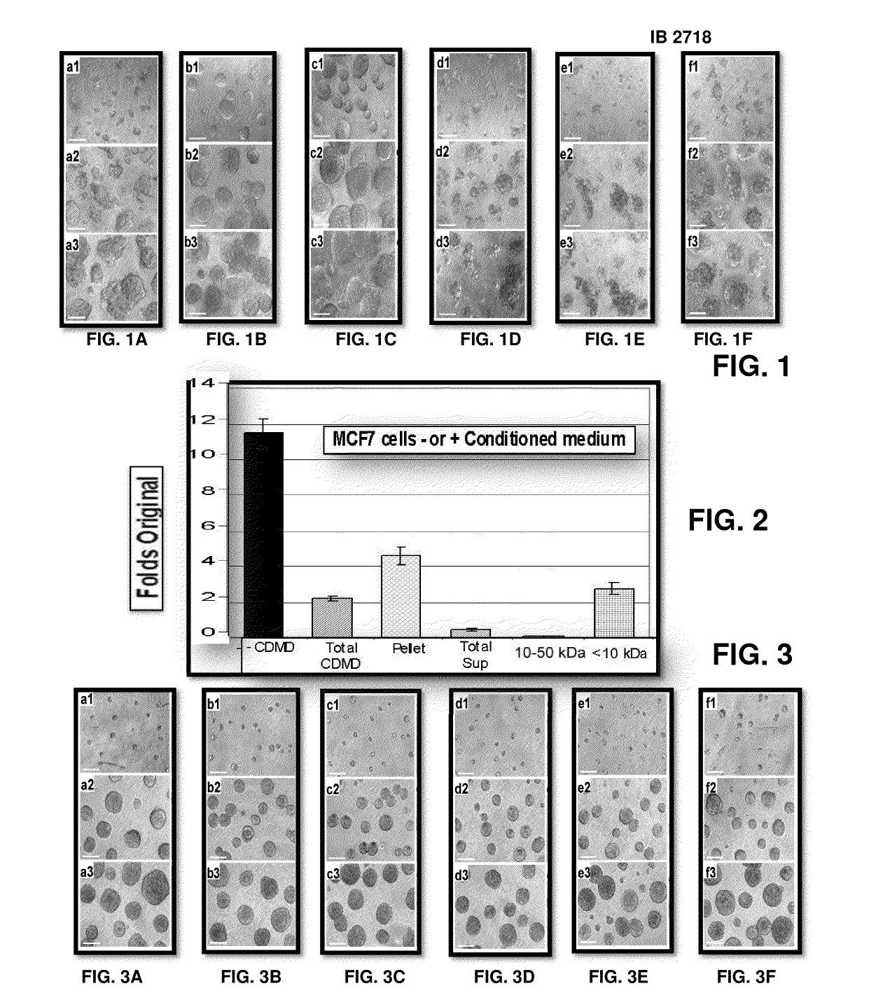

In order to determine if certain secreted factors from MEC could suppress proliferation and specifically kill breast cancer cells, the following example was conducted. Initially, CDMD from MECs was fractionated according to the solubility and molecular weight using Millipore Centricon 50 and 10 (Fisher). Each fraction was supplemented with 2% Matrigel and growth factors as described for MCF10A growth medium (Debnath et al., 2003) and then applied separately to recipient MCF7 cells seeded in eight-well chamber slides coated with Matrigel. Morphologies of cells fed with fractions containing total supernatant (c), 10-50 kDa (d), <10 kDa (e), were carefully monitored over a week using confocal microscopy. Surprisingly, the 10-50 kDa fraction, but not the other fractions, showed a killing activity on MCF7 cells (FIG. 1). On day 7, Matrigel was digested by dispase and released acinus structures were digested by trypsin to give individual cells. Viable cell numbers were counted using trypan blue staining and the fold increase compared to the original cell number seeded was calculated. The number of viable cells cultured with the 10-50 kDa fraction dropped to being undetectable after a week (FIG. 2). As a control, fractions of the CDMD prepared in the same way were applied to recipient MCF10A cells for a week, and no cytotoxicity was observed (FIG. 3). These results indicate that certain secreted factors suppress proliferation and specifically kill breast cancer cells. The above data was based on MCF7 cells which were derived from a pleural effusion containing metastatic tumor cells from a human mammary adenocarcinoma (Soule et al., 1973). MCF7 cells retain several characteristics of differentiated MECs including ability to process 17β-estradiol (E2) via cytoplasmic estrogen receptors (ERs) (Brandes and Hermonat, 1983; Sugarman et al., 1985). To exclude the possibility of cell type specificity, the cytotoxic activity of the fractionated medium on other types of breast cancer cell lines including SKBR3, MDA-MB-231 and MDA-MB-468, was tested following the same experimental procedures as with MCF7 cells. The results confirmed that the 10-50 kDa fraction of CDMD exerts cytotoxic activity on a broad spectrum of breast cancer cell lines (data not shown).

### Example 2

### Identification of Cytotoxic Secreted Factors in the Conditioned Medium

This example describes the identification of factors secreted by MCF10A cells (cytotoxic factors) that are not secreted by MCF7 cells (no cytoxocity in CDMD from these cells). To identify the differentially secreted proteins, proteomics mass spectrometry was performed to analyze the CDMD collected from MCF10A and MCF7 cells cultured in Matrigel. To identify the differentially secreted proteins, the CDMD was collected from MCF10A and MCF7 cells cultured in Matrigel every 12 hours for a week and the proteins were fractionated by centricon cutoff. The 10-50 kDa fraction (which exhibits the major killing activity) was collected and concentrated by trichloric acid (TCA) precipitation. The protein pellet was then subjected to SDS-PAGE gel electrophoresis to enrich the secreted factors with similar molecular weights for comparison. The gel was sliced every 2 mm and proteins in each slice were digested with trypsin. Digested peptides were eluted from gel and subjected to mass spectrometric analysis using two-dimensional liquid chromatography (strong cation exchange (SCX) as 1st dimension, reverse phase liquid chromatography (RPLC) as 2nd dimension) on-line interfaced with a quadruple-orthogonal time-of-flight tandem mass spectrometer (QSTAR XL) (Allen et al., 2002) at UCI core facilities directed by Dr. Lan Huang. The acquired spectra were submitted for automated database searching using both Mascot (http://www.matrixsciences.com) and Protein Prospector (http://prospector.ucsfedu). Hundreds of proteins were identified with a significant abundance in the 10-50 kDa fraction of the CDMD, most of which are membrane proteins. Comparative analyses of proteins secreted from MCF10A vs. MCF7 cells revealed differentially expressed factors such as interleukins (ILs), bone morphogenic proteins (BMP10), fibroblast growth factors (FGF11) and other cytokines which are involved in cell growth and death. Two anti-angiogenic factors identified were ATIII (antithrombin 3) and VDBP (Vitamin D binding protein); two pro-inflammatory factors identified were IL-1F7 and IL-25, and two growth/differentiation factors identified were FGF11 (fibroblast growth factor 11) and BMP10 (bone morphogenic protein 10).

### Example 3

### Confirmation of Cytotoxic Activity of Identified Factors

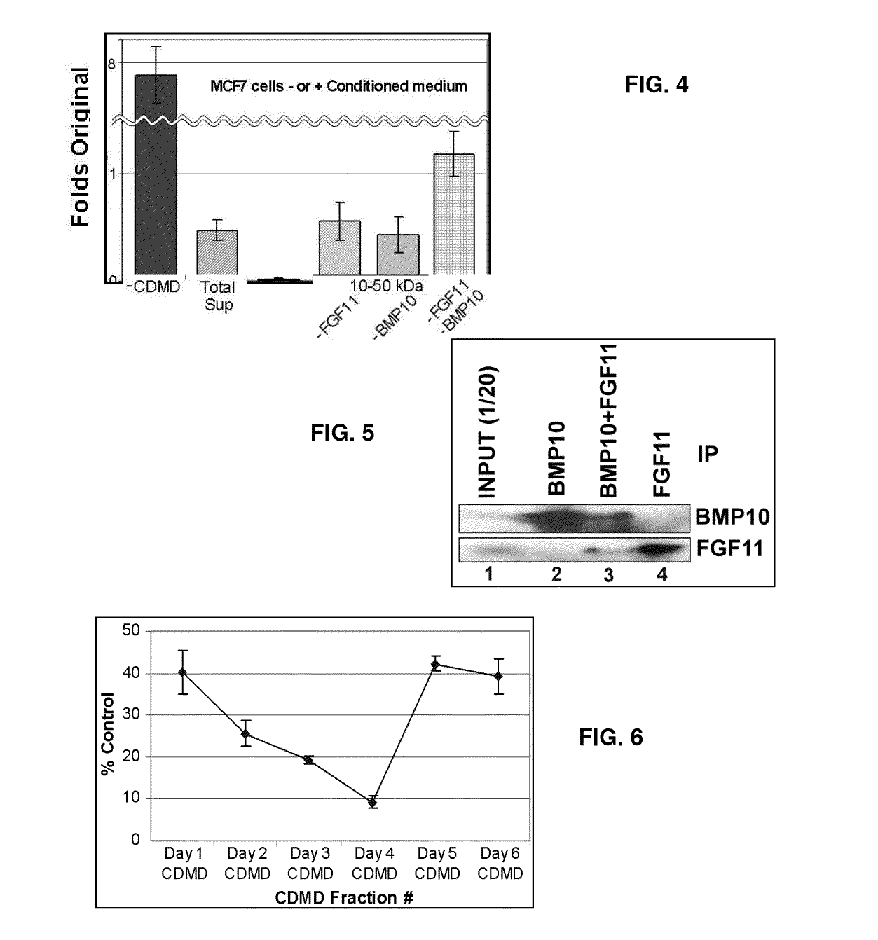

This example describes analyses to further validate the mass spectrometry data and to verify cytotoxic factors contribution to the cell killing activity. Based on the killing activity over a week (FIG. 4), media collected on days 3 and 4 with the highest killing activity were pooled for fractionation and depletion. To immuno-deplete the candidate protein, pooled media were incubated with antibodies against the target protein or control rabbit-against-mouse antibody in the presence of protein A/G agarose beads (Skildum et al., 2002). The two factors tested first using commercially available antibodies were FGF11 and BMP10. To confirm the efficacy of depletion, control and target protein-depleted media, as well as the immunoprecipitates, were analyzed by Western blot using the same antibody against the target protein (FIG. 5). The supernatant was saved for activity test in 3-D matrix. Reduction or decrease of the cell killing activity is indicative of this factor as a functional component in the cytotoxic fraction. The results indicate that both FGF11 and BMP10 are important cytotoxic factors essential factors since their depletions diminished the cytotoxic activity (FIG. 6).

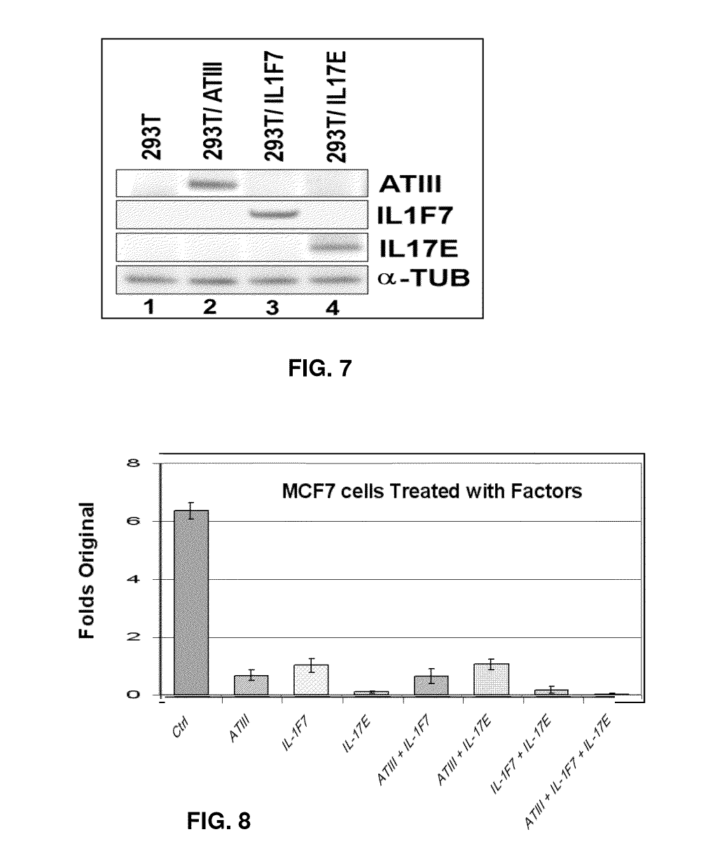

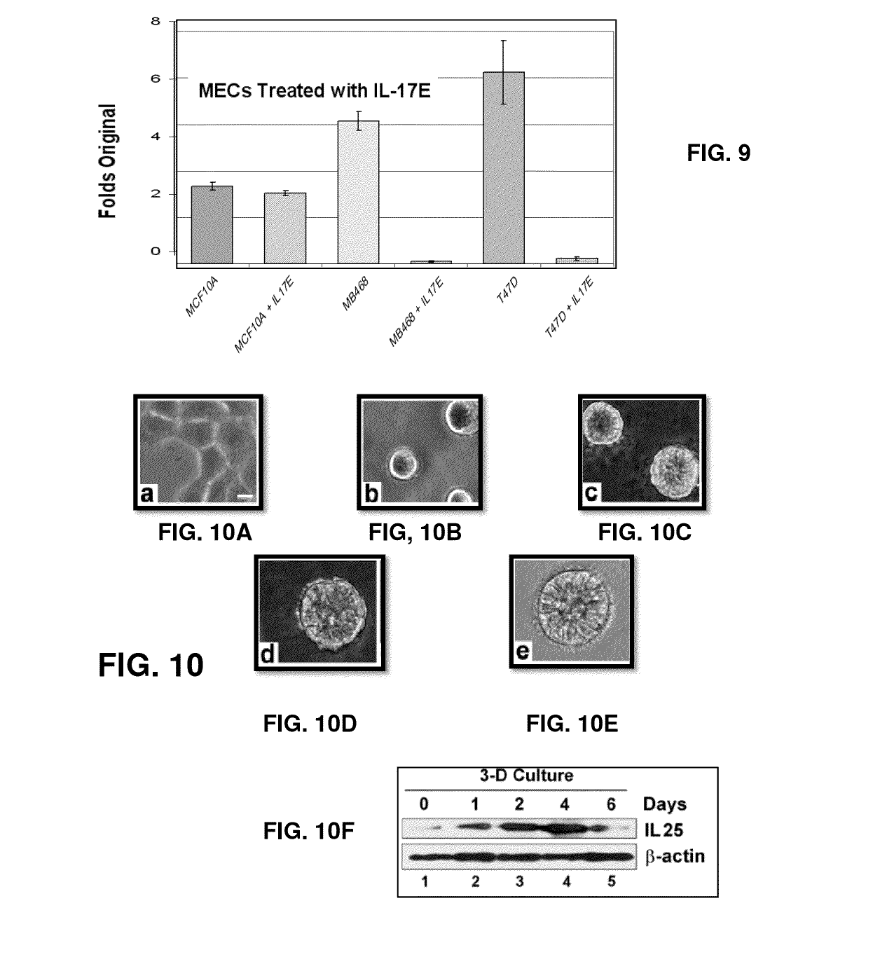

To test whether a particular factor or a group of factors is sufficient for cytotoxic activity, these factors, either individually or in combination, were directly added to breast cancer cells to determine the cell killing activity. 293T cell lines were established that stably express ATIII, IL1F7 or IL17E after hygromycin selection (FIG. 7). The secreted factors were collected in CDMD and enriched by size fractionation with Centricons 10 and 50. The factors individually or in combination were applied to MCF7 cells in 3-D culture and cell viability was monitored in comparison to the control sample treated with the fractionated CDMD from the wild-type 293 cells (FIG. 8). The data shows that IL17E exerts the highest cell killing activity on MCF7 cells where the viable cell number dropped to <10% of the original after one week. In contrast, the number of cells treated with either ATIII or IL1F7 retained the original level throughout the assay period. This result suggests that IL17E exhibits the most potent cytotoxic activity on breast cancer cells while ATIII and IL1F7 exert cytostatic activity on these cells. Then, the cell killing activity of IL17E was further tested on two other breast cancer cell lines, MD-MB468 and T47D, along with a normal MEC cell line MCF10A (FIG. 9). Surprisingly, IL17E eliminated almost all the breast cancer cells in a week, but not MCF 10A cells, showing that the cytotoxic activity of IL17E is specific to breast cancer cells.

It was hypothesized that IL25 (IL17e) expression in normal MECs is confined in differentiating acini (FIG. 10a-e) and therefore IL25 protein level during acinus differentiation was monitored for one week. IL25 level started to increase once cells were plated in 3-D matrix and continued to rise until the peak at day 4 (FIG. 10f), around the onset of luminal apoptosis in acini (FIG. 10a-e). This observation indicates that IL25 is temporally upregulated in MECs along with the advance of acinus differentiation. Interestingly, the expression pattern of IL25 appeared to be correlated with the cytotoxicity of CDMD that also peaked at day 4 (FIG. 4), indicating IL25 as a key component for this activity.

### Example 4

### IL25 Inhibits the Growth of Tumor Cells Both In Vitro and In Vivo

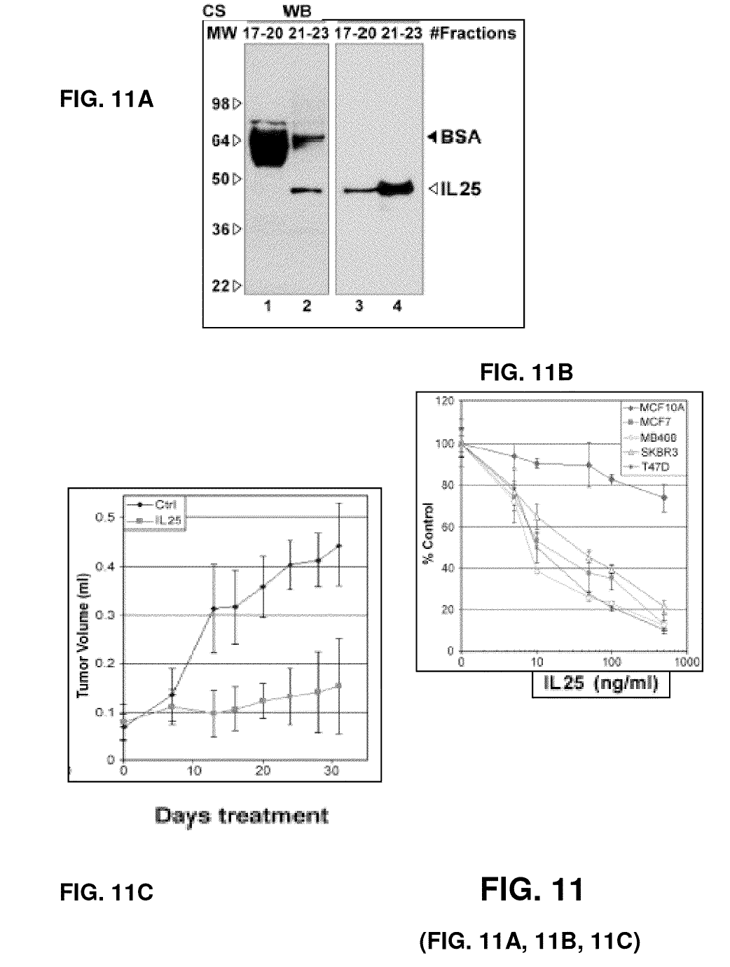

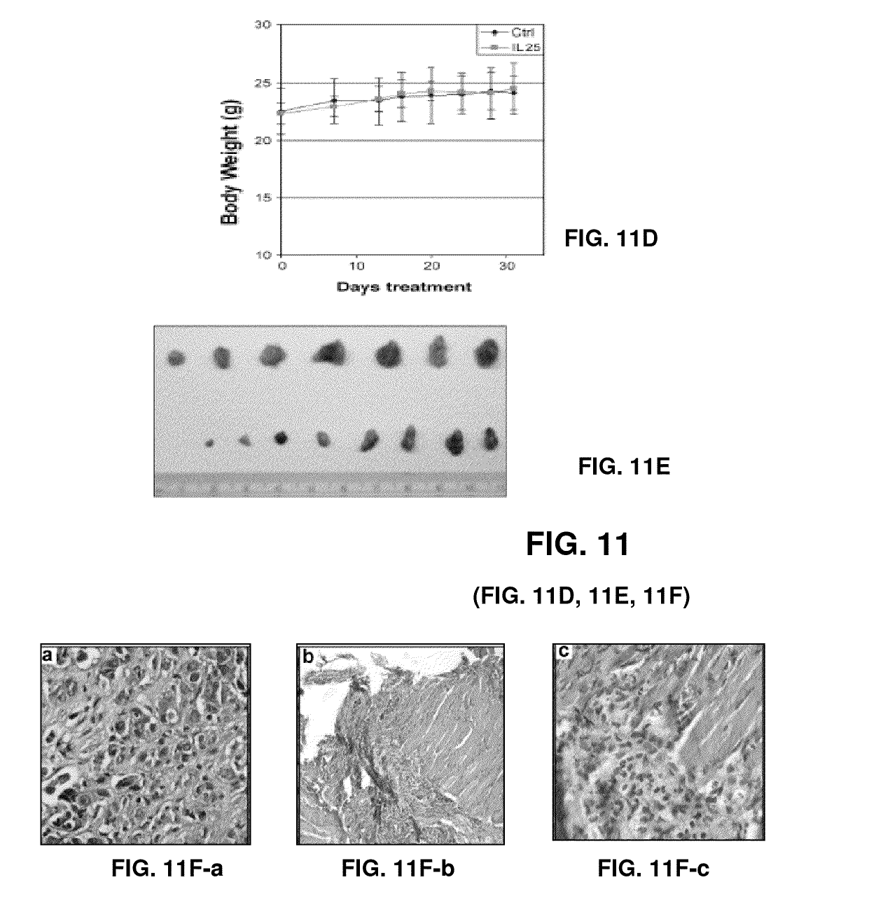

IL25 was purified from the 293T cell clone stably expressing IL25 after retroviral mediated gene transfer. Since secreted IL25 was expected to be highly glycosylated as in the case of other interleukine family members (J. K. Kolls), the total glycoproteins were affinity purified, then separated by gel filteration. On denaturing gel, glycosylated IL25 migrated at ˜48 kDa (FIG. 11A). The IL25 fraction contained a significant amount of BSA carryover from the previous peak. Nevertheless, it was decided to maintain this carrier protein to enhance the stability and function of IL25. By disregarding BSA contaminant, the purity of IL25 was estimated to be 90-95%.

To test the in vitro efficacy of purified IL25, different breast cell lines were cultured with IL25 at various doses and evaluated their viabilities by colony formation assay. Normal MEC MCF10A cells were relatively resistant to IL25. On the other hand, all the four breast cancer cell lines tested (MCF7, MDA-MB468, SKBR3 and T47D) were sensitive, showing the IC50 value of about 10 ng/ml (˜500 pM; MW=20 kDa for non-glycosylated protein) (FIG. 11B).

Next, the in vivo potency of IL25 was tested in a xenografted breast cancer model using MDA-MB468 cells growing at the mammary fat pads of nude mice. The tumors were grown for 10 days and then treated with vehicle (n=7) or IL25 (n=8, 300 ng) by on-site injection once a day for a month. As the tumor growth was monitored throughout treatment, it was determined that IL25 significantly retarded the growth of xenografted tumors. After 1 month, the average size of IL25-treated tumors was about 3 fold smaller than control tumors (p=0.0016) (FIG. 11C). In addition, as the body weight of mice was measured to assess the systemic stress caused by the treatment, there was no difference between the control and the treated group (FIG. 11D). For more stringent safety tests, 5 times higher dose of IL25 (1.5 mg) was injected into the tail veins of C57 mice (vehicle: n=3; IL25: n=5). No signs for illness (e.g., lethargy and weight loss, data not shown) were observed. Tumors were excised for pathological examinations. Comparison of the excised whole tumor masses clearly showed that IL25 treatment significantly reduced the tumor size (FIG. 11E). When the histology of the excised tumors was examined, control group displayed actively dividing tumor cells (FIG. 11F.a). Conversely, IL25-treated tumors exhibited reduced proliferative behavior with the average mitotic index 2 fold lower than control tumors (0.63±0.055 vs. 1.1±0.17, p=0.053; data not shown). Noteworthily, in one of the treated samples, the tumor was completely regressed and replaced by a crowd of macrophage infiltrates in the lesion (FIG. 11F.b,c). These observations indicates that IL25 exerts anti-tumor activity in animals by inhibiting the growth or inducing cell death of tumors.

### Example 5

### IL25 Receptor IL25R (IL17RB) is Highly Expressed in Breast Tumor Cells, but Not in Normal MECs

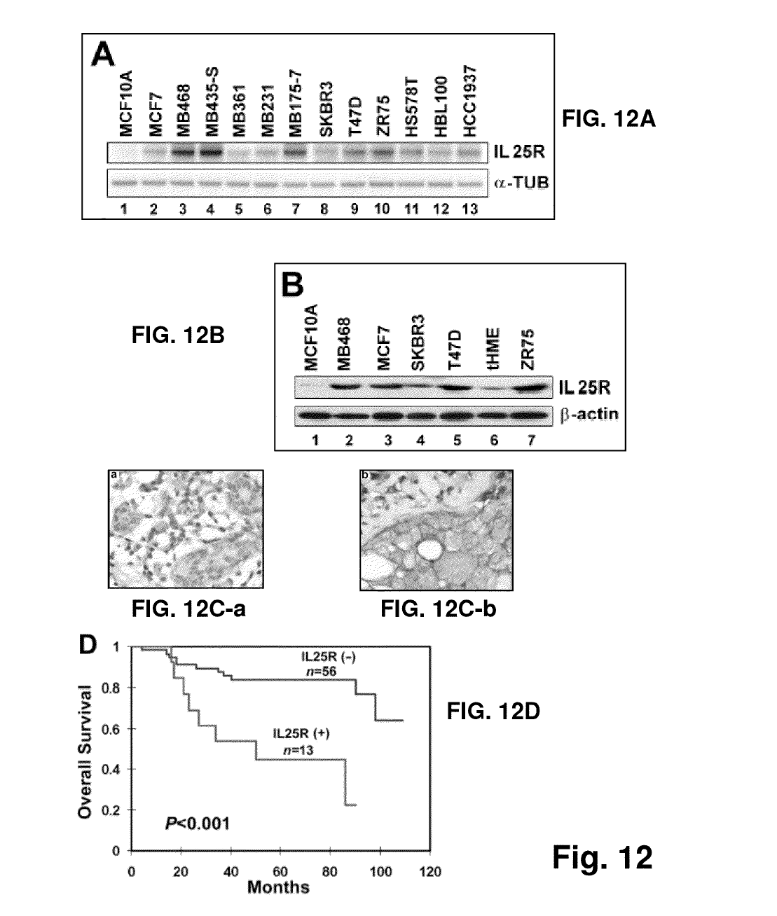

In this example, the expression of IL25R was screened in a panel of breast cell lines with various pathogenic traits (e.g., estrogen receptor (ER)-positive: MCF7, MDA-MB361, T47D and ZR75; ER-negative: MCF10A, MDA-MB468, MB435-S, MB231 and MB175-7, SKBR3, HS578T, HBL100 and HCC1937) (25). The RT-PCR result showed that IL25R was expressed at a moderate to high level in all the breast cancer cell lines tested, but expressed at a significantly lower level in MCF10A (FIG. 12A). To further confirm this observation, the protein level of IL25R was analyzed by western analysis with one additional normal cell line, telomerase-immortalized human mammary epithelia (tHME). The result confirmed that IL25R expression levels in the two normal cell lines, MCF0A and tHME, were significantly lower than those in breast cancer cell lines (FIG. 12B).

Next, the expression patter of IL25R was examined in human breast tumor specimens. In the immunoreactive samples, membranous staining pattern of IL25R was seen in cancerous cells (FIG. 12C.b), but not in nonmalignant MECs of ducts and lobules (FIG. 12C.a). Consistently, IL25R is expressed at a very low level in normal mammary tissue (Lee et al., 2001). Importantly, 18.8% of tumor specimens examined ( 13/69) displayed a clear membranous staining pattern. These positively stained tumors were correlated with poor prognosis and significantly high mortality of patients (p<0.001) (FIG. 12D).

### Example 6

### IL25 Induces the Formation of Death Complex at the Receptor to Activate Caspase-Mediated Apoptosis

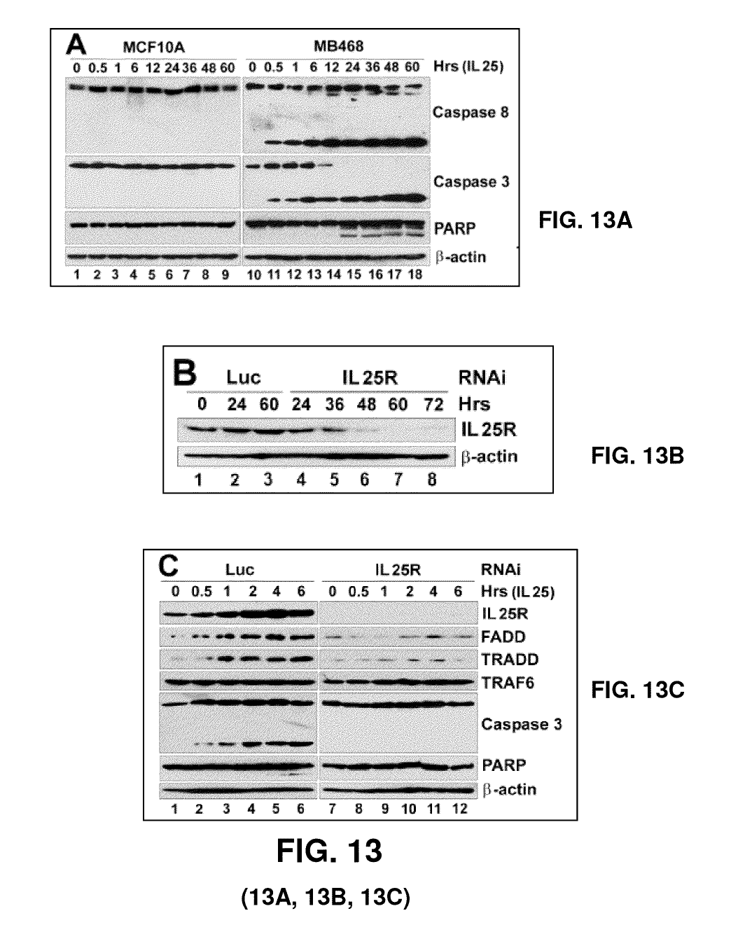

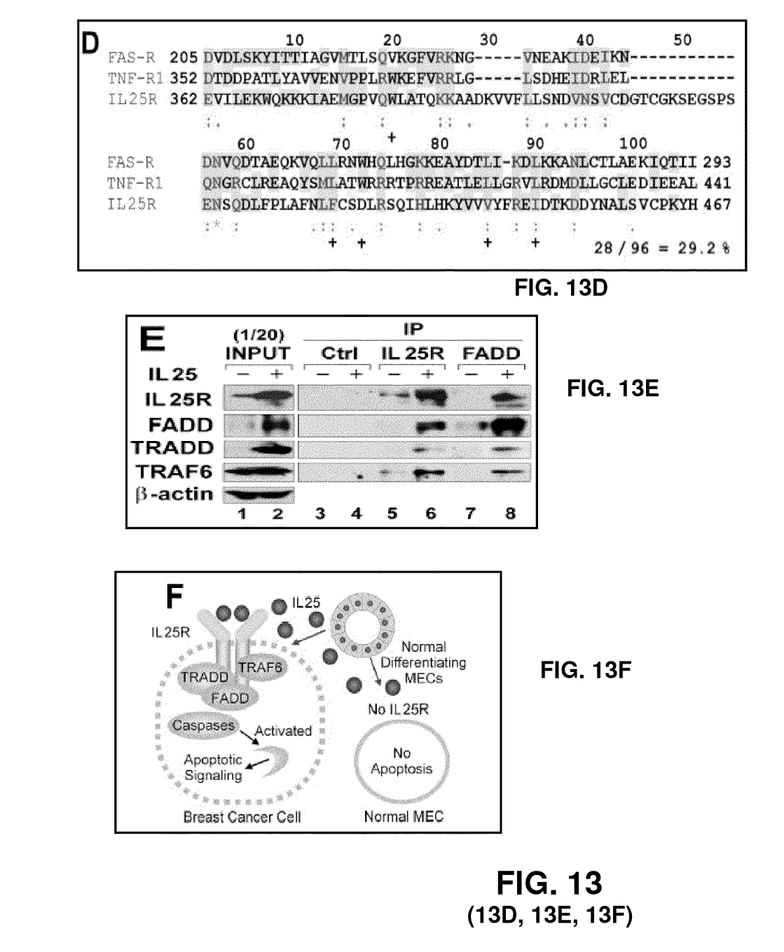

IL25 signaling via IL25R has been shown to induce pro-inflammatory response in certain tissues including lung fibroblasts (Letuve et al.). On the contrary, IL25 induces the death of breast cancer cells. To test whether IL25 treatment induces receptor-mediated apoptosis of breast cancer cells, MDA-MB468 cells were used, which express a high level of IL25R, vs. MCF10A cells, which express a low level of the receptor (FIG. 12A, 12B). In MDA-MB468 cells, IL25 caused the cleavages of caspases 8 and 3 within 30 minutes; then, the cleavage of PARP became evident after 24 hours, indicating the activation of apoptosis. Conversely, in MCF10A cells, such an apoptotic signaling was absent (FIG. 13A). This result indicates that IL25 specifically induces apoptosis of cells expressing IL25R.

To further confirm that IL25R indeed mediates death signaling for IL25, IL25R was depleted by siRNA in MDA-MB468 cells, which showed a complete loss of the protein after 60 hrs (FIG. 13B). Then, cells were treated with IL25 to test a defect in the activation of downstream effectors. In cells treated with control luciferase siRNA, IL25 induced the cleavages of caspase 3 and PARP. Conversely, in cells depleted of IL25R, these phenotypes were absent (FIG. 13C). Therefore, this result substantiates that death signal from IL25 is indeed mediated through the activation of the receptor IL25R.

If IL25 binding to the receptor can send a death signal in cells, IL25R must serve as a death receptor and contain a certain signature motif. The C-terminal region of IL25R (aa.362-467, SEQ ID NO:15) was aligned with the death domains (DDs) of FAS receptor (FAS-R: aa.205-293, SEQ ID NO:16) and TNF receptor 1 (TNF-R1: aa.352-441, SEQ ID NO:17) (FIG. 13D) and found that this region of IL25R shares about 30% similarity with both DDs. The residues highly conserved among DD-containing proteins are similar in IL25R, except for Trp72 (See the numbering in FIG. 13D) (Hofmann and Tschopp). Therefore, this region of IL25R appears to possess a DD-like motif. Interestingly, two similar receptors, IL17A and IL17B, lack such a DD-like motif. Next, it was examined how a death signal from IL25 is transduced by the receptor. If IL25R possesses a DD-like motif, it must interact with DD-associating proteins upon IL25 binding. Based on this hypothesis, MDA-MB468 cells were treated with IL25 and analyzed for the interactions of IL25R with DD adaptor proteins, FADD and TRADD (Baker and Reddy). As a positive control, the interaction of IL25R with TRAF6 was also tested. TRAF6 was shown to be constitutively associated with IL25R via a TRAF6 binding motif around Glu341 (Glu338 in mouse) of IL25R (Maezawa et al.), the region N-terminal to the putative DD-like motif (aa.362-467). It was found that IL25R strongly interacted with FADD and TRADD only in the presence of IL25, as detected by a reciprocal immunoprecipitation (FIG. 13E). Moreover, the input levels of these three proteins were largely elevated upon IL25 treatment (FIG. 13C, 13E), which might contribute to their apparently increased interactions. In contrast, TRAF6 remained associated with IL25R even in the absence of the ligand. Upon IL25 treatment, the amount of TRAF6 co-precipitated with IL25R increased in proportion to the increased IL25R level, despite that the total amount of TRAF6 did not change (FIG. 13C, 13E), suggesting that IL25R is always bound by TRAF6 as previously observed (Maezawa et al.). Taken together, these results indicate that IL25 binding to receptor increases the stability of the receptor complex containing FADD and TRADD that triggers the activation of caspase cascade resulting in apoptosis.

Interestingly, IL25R activation by IL25 in lymphoid and renal cells induces pro-inflammatory responses. This action of IL25 is mediated by the constitutively receptor-bound TRAF6 which activates NF-kB for the transcription of inflammatory cytokines (Lee et al., and Maezawa et al.). In contrast, it was found that in breast cancer cells IL25 binding to IL25R causes the receptor to interact with DD adaptor proteins, FADD and TRADD, and rapidly activates caspase-8/-3 for apoptotic signaling, despite constitutive association of TRAF6 with the receptor. Such a discrepancy may be attributed to the presence of additional proteins serving as a switch between TRAF6/NF-kB signal and TRADD/FADD/caspase-8 signal in different cellular contexts. For example, TNF-R1 activation by TNF-a induces both NF-kB activation and apoptosis; however, the former can be blocked by brain and reproductive organ expressed (BRE) protein that binds the j axtamembrane cytoplasmic region of the receptor and promotes apoptotic signaling (Gu et al.). Apparently, IL25 binding to IL25R emanates potentially diverse signaling which intricately communicates to determine the resultant output.

### Example 7

### Death Domain of IL25R is Important for Apoptotic Signaling Mediated by IL25

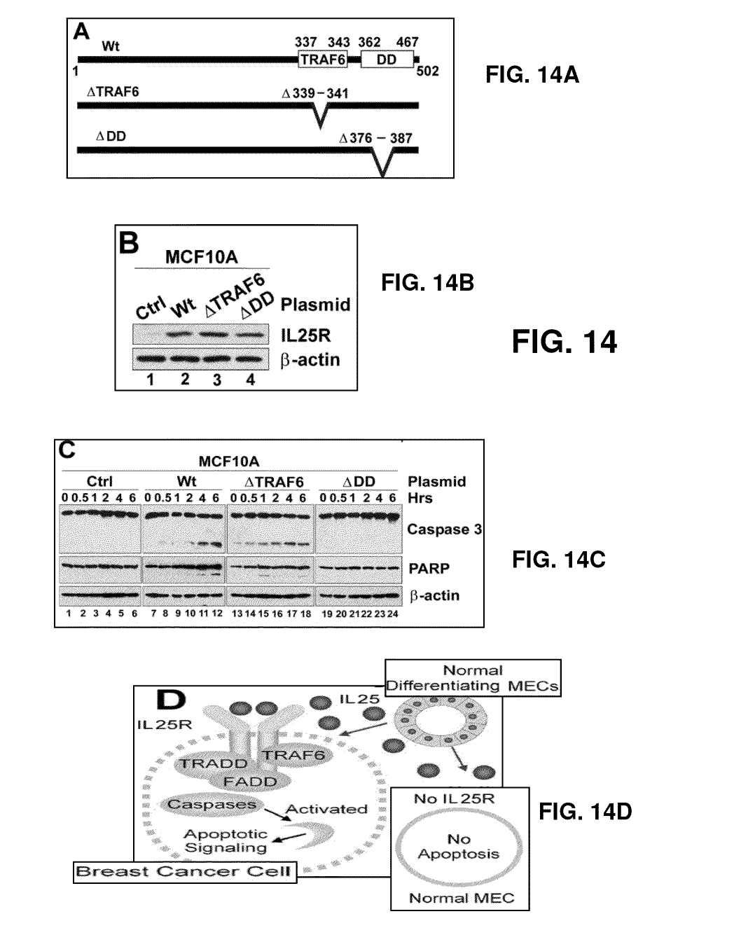

To dissect how IL25 sends death signaling via IL25R, IL25R protein, wild-type (Wt) or a deletion mutant in TRAF6 binding domain (ΔTRAF6) or DD (ΔDD) was ectopically expressed in MCF10A cells which only express a low level of the endogenous IL25R (Ctrl) (FIG. 14A, B). Upon IL25 treatment, MCF10A cells expressing wild-type IL25R displayed the apoptotic response, characterized by the cleavages of caspase 3 and PARP, in a time-dependent manner, demonstrating that IL25R expression is sufficient to render cells sensitive to death signaling of IL25. In contrast, cells expressing ΔDD mutant of IL25R was defective in such a response. This indicates that DD is essential for mediating death signal of IL25, consistent with the increased association of IL25R with DD adaptor proteins, FADD and TRADD, upon IL25 treatment. Surprisingly, cells expressing ΔTRAF6 mutant of IL25R showed an enhanced basal level of apoptosis even in the absence of IL25 stimulation, suggesting that TRAF6 binding to IL25R confers a protective effect on cells (FIG. 14C).

All publications and patents mentioned in the above specification are herein incorporated by reference. Various modifications and variations of the described compositions, methods, systems, and kits of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in art are intended to be within the scope of the following claims.

### REFERENCES

All of the following references are herein incorporated by reference in their entireties for all purposes.

Allen, N. P., Patel, S. S., Huang, L., Chalkley, R. J., Burlingame, A., Lutzmann, M., Hurt, E. C., and Rexach, M. (2002). Deciphering networks of protein interactions at the nuclear pore complex. Mol Cell Proteomics 1, 5145-5152.

Bissell, M. J., and LaBarge, M. A. (2005). Context, tissue plasticity, and cancer: Are tumor stem cells also regulated by the microenvironment? Cancer Cell 7, 17-22.

Brandes, L. J., and Hermonat, M. W. (1983). Receptor status and subsequent sensitivity of subclones of MCF-7 human breast cancer cells surviving exposure to diethylstilbestrol. Cancer Res 43, 2831-2835.

Castaigne, S., Chomienne, C., Daniel, M. T., Ballerini, P., Berger, R., Fenaux, P., and Degos, L. (1990). All-trans retinoic acid as a differentiation therapy for acute promyelocytic leukemia. I. Clinical results. Blood 76, 1385-1393.

Debnath, J., Muthuswamy, S. K., and Brugge, J. S. (2003). Morphogenesis and oncogenesis of MCF-10A mammary epithelial acini grown in three-dimensional basement membrane cultures. Methods 30, 256-268.

Furuta, S., Jiang, X., Gu, B., Cheng, E., Chen, P. L., and Lee, W. H. (2005). Depletion of BRCA1 impairs differentiation but enhances proliferation of mammary epithelial cells. Proc Natl Acad Sci USA 102, 9176-9181.

Huang, M. E., Ye, Y. C., Chen, S. R., Chai, J. R., Lu, J. X., Zhao, L., Gu, L. J., and Wang, Z. Y. (1988). Use of all-trans-retinoic acid in the treatment of acute promyelocytic leukemia. Blood 72, 567-572.

Mintz, B., and Illmensee, K. (1975). Normal genetically mosaic mice produced from malignant teratocarcinoma cells. Proc Natl Acad Sci USA 72, 3385-3589.

Skildum, A. J., Mukherjee, S., and Conrad, S. E. (2002). The cyclin-dependent kinase inhibitor p21WAF1/Cipl is an antiestrogen-regulated inhibitor of Cdk4 in human breast cancer cells. J Biol Chem 277, 5145-5152.

Soule, H. D., Vazguez, J., Long, A., Albert, S., and Brennan, M. (1973). A human cell line from a pleural effusion derived from a breast carcinoma. J Natl Cancer Inst 51, 1409-1416.

Sternlicht, M. D., Lochter, A., Sympson, C. J., Huey, B., Rougier, J. P., Gray, J. W., Pinkel, D., Bissell, M. J., and Werb, A. (1999). The stromal proteinase MMP3/stromelysin-1 promotes mammary carcinogenesis. Cell 98, 137-146.

Sugarman, B. J., Aggarwal, B. B., Hass, P. E., Figari, I. S., Palladino, M. A. J., and Shepard, H. M. (1985). Recombinant human tumor necrosis factor-alpha: effects on proliferation of normal and transformed cells in vitro. Science 230, 943-945.

Uhr, J. W., Scheuermann, R. H., Street, N. E., and Vitetta, E. S. (1997). Cancer dormancy: opportunities for new therapeutic approaches. Nat Med 3, 505-509.

Warrell, R. P. J., Frankel, S. R., Miller, W. H. J., Scheinberg, D. A., Itri, L. M., Hittelman, W. N., Vyas, R., Andreeff, M., Tafuri, A., and Jakubowski, A. (1991). Differentiation therapy of acute promyelocytic leukemia with tretinoin (all-trans-retinoic acid). N Eng J Med 324, 1385-1393.

Pierce and Speers, Cancer Res. 48, 1996-2004 (1988).

Uhr et al., Nat. Med. 3, 505-509 (1997).

Huang et al., Blood 72, 567-572 (1988).

Warrell et al., N. Eng. J. Med. 324, 1385-1393 (1991).

Dexter and Hager, Cancer 45 (Suppl.), 1178s-1184s (1980).

Zanardi et al., Endocr. Relat Cancer. 13, 51-68 (2006).

Dexter, et al., Cancer Res. 37, 3136-3140 (1977).

Walrath, et al., Occup. Med. 31, 432-438 (1989).

Russo et al., Clin. Cancer Res. 11, 931s-936s (2005).

MacMahon et al., Bull. World Health Organ. 43, 209-221 (1970).

Lambe et al., Breast Cancer Res Treat. 38, 305-311 (1996).

Janssens et al., Mol. Cell Endocrinol. 269, 93-98 (2007).

Alvarado, et al., In Vitro Cell Dev. Biol. Anim. 30A, 4-8 (1994).

Debnath, et al., Methods 30, 256-268 (2003).

Furuta et al., Proc. Natl. Acad. Sci. USA 102, 9176-9181 (2005).

Debnath et al., Cell 111, 29-40 (2002).

Wang et al., Biochemistry 46, 3553-3565 (2007).

Cao, et al., Am. J. Pathol. 161, 2053-2063 (2002).

Kalkunte, et al., Angiogenesis 8, 349-360 (2005).

Lee et al., J. Biol. Chem. 276, 1660-1664 (2001).

Kumar et al., Cytokine 18, 61-71 (2002).

Smallwood et al., Proc. Natl. Acad. Sci. USA 93, 9850-9857 (1996).

Chen et al., Development 131, 2219-2231 (2004).

Kolls, A. Linden, Immunity 21, 467-576 (2004).

Neve et al., Cancer Cell 10, 515-527 (2006).

Letuve et al., J. Allergy. Clin. Immunol. 117, 590-596 (2006).

Hofmann, et al., FEBS Lett. 371, 321-323 (1995).

Baker, et al., Oncogene 17, 3261-3270 (1998).

Maezawa et al., J. Immunol. 176, 1013-1018 (2006).

Gu, et al., FASEB 12, 1101-1108 (1998).

Piccirillo et al., Nature 444, 761-765 (2006).

Chalkley et al., Mol. Cell Proteomics 4, 1194-1204 (2005).

Furuta et al., Cancer Cell 10, 13-24 (2006).

We claim:

1. A method comprising contacting breast cancer cells with a therapeutic amount of at least one cytotoxic factor selected from the group consisting of: IL-25, BMP10, FGF11, and VDBP.

2. The method of claim 1, wherein the cytotoxic agent suppresses proliferation of the breast cancer cells.

3. The method of claim 1, wherein the agent does not suppress differentiation of the breast cancer cells.

4. The method of claim 1, wherein the cytotoxic agent is IL-25.

5. The method of claim 1, wherein the cytotoxic agent is BMP10.

6. The method of claim 1, wherein the cytotoxic agent is FGF11.

7. The method of claim 1, wherein the cytotoxic agent is VDBP.

8. The method of claim 1, wherein the contacting kills at least a portion of the breast cancer cells.

9. The method of claim 1, wherein the contacting is performed in vivo or in vitro.

10. The method of claim 1, wherein the at least one cytotoxic agent includes at least two of the cytotoxic agents.

11. The method of claim 1, wherein the at least one cytotoxic agent includes at least 3 or all 4 of the cytotoxic agents.

12. A composition comprising:
a) a known breast cancer treatment agent and
b) at least one cytotoxic agent selected from the group consisting of: IL-25, BMP10, FGF11, and VDBP.

13. A kit comprising
a) a known breast cancer treatment agent and
b) at least one cytotoxic agent selected from the group consisting of: IL-25, BMP10, FGF11, and VDBP.

14. A method comprising: treating breast cancer cells with a size fractioned conditioned medium (CDMD) collected from differentiating normal MECs, wherein the size fractioned conditioned medium is enriched for the 10-50 kDa fraction.

15. The method of claim 14, wherein the size fractioned condition medium is enriched at least 2-fold compared to un-enriched conditioned medium.

16. The method of claim 14, wherein the breast cancer cells are differention-defective.
